(19) United States
(12) Patent Application Publication
(10) Pub. No.: US 2025/0163500 A1
(43) Pub. Date: May 22, 2025

(54) METHODS AND DEVICES FOR SPATIALLY ENCODED BIOLOGICAL ASSAYS

(71) Applicant: AtlasXomics Inc., New York, NY (US)

(72) Inventor: Joseph C. GENNARO, New York, NY (US)

(21) Appl. No.: 18/744,444

(22) Filed: Jun. 14, 2024

Related U.S. Application Data
(63) Continuation of application No. 17/466,911, filed on Sep. 3, 2021, now abandoned.
(60) Provisional application No. 63/074,764, filed on Sep. 4, 2020.

(51) Int. Cl. C12Q 1/6832

(52) U.S. Cl. CPC C12Q 1/6832

(57) ABSTRACT

The present disclosure generally relates to methods for increasing the region of interest of spatial multi-omics techniques while retaining single-cell resolution. These methods can improve the scaling of region of interest dimension with input/output channels from linear to super-linear. In some embodiments, the method is performed by providing a plurality of probes of a first type to a first region of a sample, wherein at least a subset of the probes of the first type includes a first spatial barcode, providing a plurality of probes of a second type to a second region of the sample, wherein at least a subset of the probes of the second type includes a second spatial barcode, and providing a probe of a third type to a third region of the sample, wherein the probe of the third type comprises a third spatial barcode.

### RELATED APPLICATIONS

This application is a continuation of U.S. Non-Provisional application Ser. No. 17/466,911, filed Sep. 3, 2021, entitled “METHODS AND DEVICES FOR SPATIALLY ENCODED BIOLOGICAL ASSAYS” which claims the benefit of U.S. Provisional Application Ser. No. 63/074,764, filed Sep. 4, 2020, entitled “METHODS AND DEVICES FOR SPATIALLY ENCODED BIOLOGICAL ASSAYS,” which is hereby incorporated by reference, in its entirety.

### FIELD

The present disclosure relates generally to methods and devices for spatially encoded biological assays.

### BACKGROUND

The spatial location of molecules in a sample can be identified by linking one or more probes to the molecule. Some techniques for determining the location of molecules in a sample by delivering probes in a spatially-defined manner lack a practical way to increase the area sampled while retaining adequate spatial resolution. For example, some techniques lack a practical way to increase the amount of tissue sampled while retaining adequate spatial resolution to resolve the feature profiles of individual cells.

### BRIEF SUMMARY

Mapping the spatial distribution of molecules, such as molecules associated with genomic, transcriptomic, proteomic, and other features, in a sample, such as an intact tissue sample, herein referred to as “spatial multi-omics,” holds great promise for uncovering the origins of disease and improving our understanding of biological mechanisms. Maps created by these methods have enabled researchers working in diverse application areas to gain new biological understanding, for example by creating atlases of cell types to aid in the fight against cancer (Rozenblatt-Rosen O, Regev A, Oberdoerffer P, et al. The Human Tumor Atlas Network: Charting Tumor Transitions across Space and Time at Single-Cell Resolution. Cell. 2020; 181(2):236-249. doi:10.1016/j.cell.2020.03.053), exploring the role of cellular neighborhoods in tumor development, (Schürch C M, Bhate S S, Barlow G L, et al. Coordinated Cellular Neighborhoods Orchestrate Antitumoral Immunity at the Colorectal Cancer Invasive Front [published online ahead of print, 2020 Jul. 31]. Cell. 2020; 50092-8674(20)30870-9. doi:10.1016/j.cell.2020.07.005), and other key features of diseased tissue organization, including the spatial patterning of genes with respect to non-cell features such as plaques (Wei-Ting Chen, Ashley Lu, Katleen Craessaerts, Joakim Lundeberg, Mark Fiers, Bart De Strooper, Spatial Transcriptomics and In Situ Sequencing to Study Alzheimer's Disease, Cell. 2020:Volume 182, Issue 4, 20 Aug. 2020, Pages 976-991.e19, doi:10.1016/j.cell.2020.06.038), between neurons, glial cells, and blood vessels, and tumor micro-environments (Keren et al, A Structured Tumor-Immune Microenvironment in Triple Negative Breast Cancer Revealed by Multiplexed Ion Beam Imaging, Cell:2020, Volume 174, Issue 6, 6 Sep. 2018, Pages 1373-1387.e19, doi: 10.1016/j.cell.2018.08.039), and tumor organization viewed through both mRNA and protein profiling (Merritt, C. R., Ong, G. T., Church, S. E. et al. Multiplex digital spatial profiling of proteins and RNA in fixed tissue. Nat Biotechnol 38, 586-599 (2020), doi.org/10.1038/s41587-020-0472-9).

Various spatial multi-omics tools are available, both commercial and open-source. They can be characterized by a range of capabilities including multiplexing (i.e., the type and number of different molecular species that can be imaged), spatial resolution, sensitivity (i.e., the likelihood that a molecule, such as a target molecule, present in the sample will be successfully detected), and readout modality (microscopic vs. Next-Generation Sequencing-based). Limitations of these tools can include experimental cost (e.g. on a per-assay or per-species basis), limited availability of appropriate experimental and control tissue samples, available equipment and personnel to successfully perform the experiment (if not provided as a service) and time (both hands-on and overall turnaround time).

Areas of interest in a sample, such as a tissue section, can display an irregular shape and/or have a large size. For example, tumor microenvironments, a key research focus of spatial multi-omics, can range in size from fractions of a millimeter to ˜centimeters in diameter or length. Optical-based methods such as smFISH or various in-situ sequencing (ISS) methods (e.g., FISSEQ, Cartana In Situ Sequencing) can image large areas of tissue at sub-cellular resolution, but the experimental cost and microscope time required can be proportional to both the number of types of target species and the area of tissue to be assayed. Other techniques such as Nanostring's GeoMX DSP tool can allow users to customize regions of interest (ROI's) based on investigation of adjacent sections from the same tissue block; however this technique's throughput in terms of area of tissue assayed per unit time is inversely proportional to the area of the regions of interest, and using it to profile a large area of tissue at single-cell resolution (e.g., approximately 10-25 microns or smaller) can be prohibitively costly and time-consuming (Merritt, C. R., Ong, G. T., Church, S. E. et al. Multiplex digital spatial profiling of proteins and RNA in fixed tissue. Nat Biotechnol 38, 586-599 (2020), doi.org/10.1038/s41587-020-0472-9).

Next-generation sequencing (NGS)-based methods show particular promise as discovery vehicles by combining un-biased, highly multiplexed capabilities (e.g., pan-transcriptome or high-plex protein panels) and accessibility (since they can leverage core NGS services rather than in-house microscopes and microscopy expertise, and NGS costs and turnaround time are projected to continue decreasing rapidly (Hayden, Nature 507, 294-295 (20 Mar. 2014) doi:10.1038/507294a, www.nature.com/news/technology-the-1-000-genome-1.14901). Solid-phase barcode capture array-based techniques (e.g., ST, HDST, Visium, Slide-seq) utilize capture probes confined to printed features or custom-manufactured microbeads, but not all achieve single-cell resolution (e.g., ST, Visium), and those that can are not commercially available and can have low capture efficiency (e.g., Slide-seq, HDST).

A microfluidics-based reagent delivery based platform called DBiT-seq (Deterministic Barcoding in Tissue for spatial omics sequencing) features single-cell resolution, sufficient capture efficiency for high-quality data analysis and simultaneous pan-transcriptome and protein-panel profiling (see, e.g., Liu et al. (2020). High-Spatial-Resolution Multi-Omics Atlas Sequencing of Mouse Embryos via Deterministic Barcoding in Tissue. doi: 10.1101/788992). However, the size of its region of interest (“ROI”) can be limited to 1×1 millimeter at 10 micron spatial resolution, or 2.5×2.5 millimeters at 25 micron resolution. While this can be sufficient for some applications, there remains a need for the ability to analyze larger regions of a sample without sacrificing spatial resolution. For example, there remains a need on the part of many end users, including those investigating immune tumor microenvironments (iTMEs) (Gerdes M J, Sood A, Sevinsky C, Pris A D, Zavodszky M I and Ginty F (2014) Emerging understanding of multiscale tumor heterogeneity. Front. Oncol. 4:366. doi: 10.3389/fonc.2014.00366) oncogenesis and its relationship to novel cell types, and other cutting-edge research avenues to simultaneously profile larger (e.g. 3×3 mm or greater, 1×1 cm or greater) regions of tissue sections without sacrificing single-cell spatial resolution.

The DBiT-seq technique can build spatial barcodes in situ by delivering one or more probes to overlapping areas, for example, by delivery through channels created by two microfluidic chips. For example, in one embodiment described in Liu et al. (2020). High-Spatial-Resolution Multi-Omics Atlas Sequencing of Mouse Embryos via Deterministic Barcoding in Tissue. doi: 10.1101/788992 a pair of microfluidic devices are affixed in series to a sample, such as a tissue section, thereby exposing areas of the sample (e.g., a tissue) to rectangular channels (e.g., 10 micron) overlapping at right angles. This segments the sample into discrete spatial areas (e.g., squares with edge lengths of 10×10 microns), inside of which all the barcodes (synthesized in situ) display a certain sequence or pattern encoding the spatial location of the region. By analogy with the contraction of “picture elements” to “pixels” in digital images, herein these discrete elements, when used with reference to a tissue sample, are referred to as “tixels”. However, as used herein, reference to “tixels” is not meant to limit the sample to a tissue sample. In order to scale the region of interest of such a design, one can pattern more and more microfluidic channels onto each of the two chips, with the linear dimension characterizing the region of interest scaling linearly with the number of channels (See FIG. 6 for a quantitative summary of this scaling behavior). Logistical difficulties (e.g., liquid handling, cost of unique reagents, occurrence of reagent loading errors and flow blockages, etc.) can also grow linearly with the number of channels. Furthermore, expansion of the chip footprint (its length and width), to accommodate the larger number of channels and input/outlet ports, can exacerbate known challenges with typical microfluidic chip materials, such as polydimethylsiloxane (PDMS), which can shrink upon curing (Madsen et al, Accounting for PDMS shrinkage when replicating structures, Journal of Micromechanics and Microengineering, Volume 24, Number 12, iopscience.iop.org/article/10.1088/0960-1317/24/12/127002/meta) by a temperature-dependent amount. Since the shrinkage scales with device length, the resulting loss of precision can be greater in chips with larger footprints. While it can be technically possible to use more space-efficient liquid delivery methods (such as in-molded pins fed by tubes rather than wells), it is preferable from a practical standpoint to adhere to industry standards for well spacing (American National Standards Institute: Well Positions for Microplates, published Oct. 13, 2011, slas.org/SLAS/assets/File/ANSI_SLAS_4-2004_WellPositions.pdf, accessed Sep. 3, 2020). Devices compliant with this standard can permit the use of economically-scalable liquid handling methods such as automated liquid handlers and multi-channel pipettes, which can be designed largely with such industry standards in mind.

Accordingly, the present technique provides methods and devices in which a linear dimension of region of interest of a sample can be increased without a corresponding increase in the number of channels and/or expansion of a device footprint.

In accordance with some embodiments, a method is described. The method includes: providing a plurality of probes of a first type to a first region of a sample, wherein at least a subset of the probes of the first type includes a first spatial barcode; linking at least a first probe of the probes of the first type to a molecule in the sample; providing a plurality of probes of a second type to a second region of the sample, wherein: at least a subset of the probes of the second type includes a second spatial barcode different from the first spatial barcode; and the first and second regions intersect at two or more noncontiguous locations on the sample; linking a first probe of the probes of the second type to the first probe of the probes of the first type at a first intersection of the first and second regions; providing a probe of a third type to a third region of the sample, wherein the probe of the third type comprises a third spatial barcode different from the first and second spatial barcodes; linking the probe of the third type to the first probe of the first type and/or the first probe of the second type; and identifying that the molecule is present in the sample at the first intersection based on at least the first, second, and third spatial barcodes. In some examples, the method includes identifying that the molecule is present at an intersection of the first, second, and third regions. In some examples, a plurality of probes of the probes of the third type can be provided, wherein at least a subset of the probes of the third type includes a third spatial barcode different from the first and second spatial barcodes. In some embodiments, the methods provided herein include identifying the molecule.

Thus, methods and devices are provided with a larger region of interest dimension, while minimizing device footprint, without loss of resolution. Such methods may complement or replace other methods and devices for spatially encoded biological assays. In some embodiments, the methods provided herein can be used to perform one or more analyses selected from among transcriptomic, epigenomic, genomic, epigenetic, genetic, proteomic, bioinformatic and panomic analysis. In some embodiments, the methods provided herein can identify intratumor heterogeneity. In some embodiments, intratumor heterogeneity can be identified by epigenetic profiling.

### DESCRIPTION OF THE FIGURES

The patent or application file contains at least one drawing executed in color. Copies of this patent or patent application publication with color drawing(s) will be provided by the Office upon request and payment of the necessary fee.

For a better understanding of the various described embodiments, reference should be made to the Description of Embodiments below, in conjunction with the following drawings in which like reference numerals refer to corresponding parts throughout the figures.

FIG. 1 illustrates two intersections that can be distinguished by an additional spatial barcode in accordance with some embodiments.

FIG. 2 illustrates twelve intersections that can be distinguished by an additional spatial barcode in accordance with some embodiments.

FIG. 3 is a diagram of an exemplary barcoding strategy via multiple ligation in accordance with some embodiments.

FIG. 4 is a flow diagram illustrating a method for a spatially encoded biological assay in accordance with some embodiments.

FIGS. 5A and 5B illustrate delivery of four sets of barcodes A1-A3 and B1-B3 (FIG. 5A) and C1-C50 and D1-D50 (FIG. 5B) in accordance with some embodiments.

FIG. 6 is a chart comparing exemplary device footprints that can achieve a region of interest area with microfluidic channels of 10 micron width, in units normalized to a standard 25×75 mm histology slide.

FIG. 7 illustrates a 156×156 microfluidic device with dimensions of 10 cm×6 cm with a 3 mm×3 mm region of interest.

FIG. 8 illustrates a device containing a reagent deposition window in accordance with some embodiments.

FIGS. 9A and 9B illustrate a device with a 9 mm² region of interest. The device includes a chip with a first group of channels, a chip with a second group of channels, and a chip with a 3×3 grid of windows (FIG. 9A), and has a 75 mm×25 mm footprint (FIG. 9B).

FIGS. 10A and 10B illustrate a device with a 36 mm² region of interest. The device includes a chip with a first group of channels, a chip with a second group of channels, and a chip with a 3×3 grid of windows (FIG. 10A), and has a 75 mm×50 mm footprint (FIG. 10B).

FIGS. 11A-11D illustrate a three-chip device on a standard histology slide footprint (75 mm×25 mm) with a segmented region of interest (ROI) in which three sets of barcodes can be delivered by microfluidic channels (FIGS. 11A, 11B, and 11C). FIG. 11D illustrates an enlargement of microfluidic channels of FIG. 11C.

FIGS. 12A and 12B illustrate a 4×4 grid of tixel groupings using a hashtagged spatial encoding matrix based on sequential bifurcation (e.g., sequential bifurcation of photoactivated barcodes). FIG. 12A illustrates a combination of photomasks illustrated in FIGS. 13A-13D.

FIG. 12B identifies regions of tixel groupings in FIG. 12A.

FIGS. 13A-13D illustrate photomasks that can produce a 4×4 grid of tixel groupings illustrated in FIGS. 12A-B.

FIG. 14 illustrate components of a three-chip device with five discrete regions of interest.

FIG. 15 depicts images of fresh-frozen human heart tissue sections.

FIGS. 16 and 17 depict data readouts of an assay performed on the tissue sections depicted in FIG. 15 using the device depicted in FIG. 14.

FIG. 18 is a flow diagram illustrating a method for a spatially encoded biological assay in accordance with some embodiments.

### DESCRIPTION OF EMBODIMENTS

The following description sets forth exemplary methods, parameters, and the like. It should be recognized, however, that such description is not intended as a limitation on the scope of the present disclosure but is instead provided as a description of exemplary embodiments.

There is a need for methods that increase the region of interest in methods in which the location of a molecule in a sample is identified, while retaining resolution. For example, there is a need for a practical method to increase the region of interest of microfluidic-based NGS spatial multi-omics solutions while retaining single-cell resolution and improving the scaling of region of interest dimension with input/output channels from linear to super-linear.

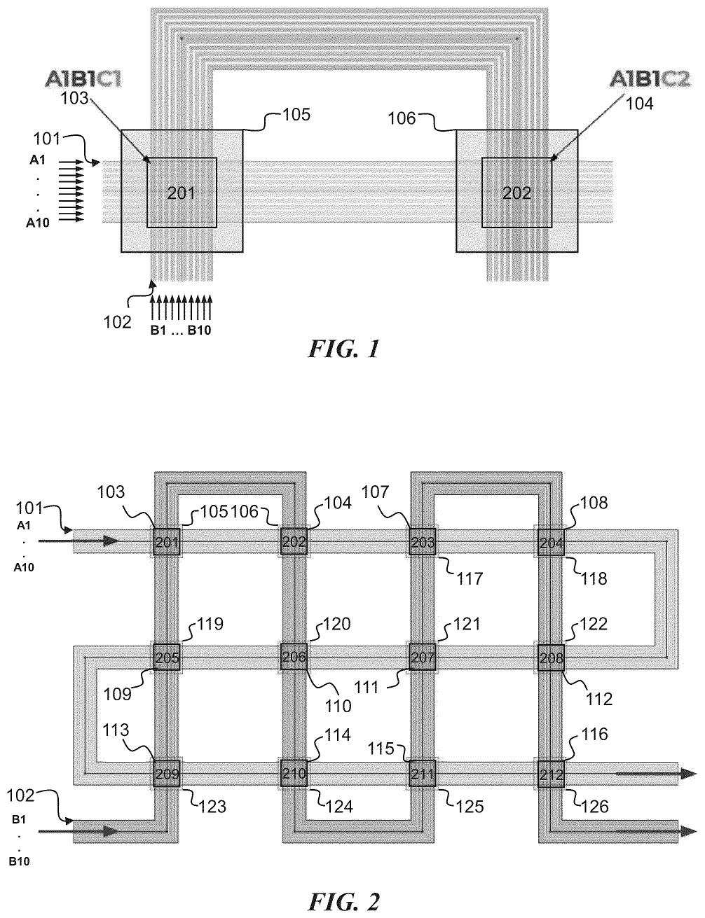

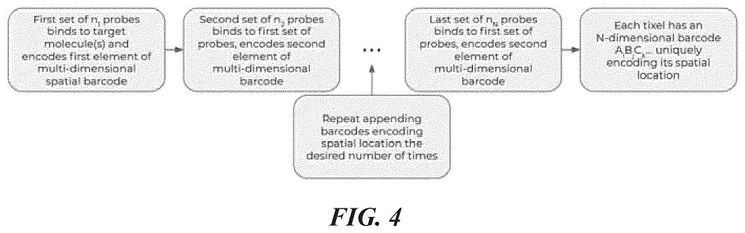

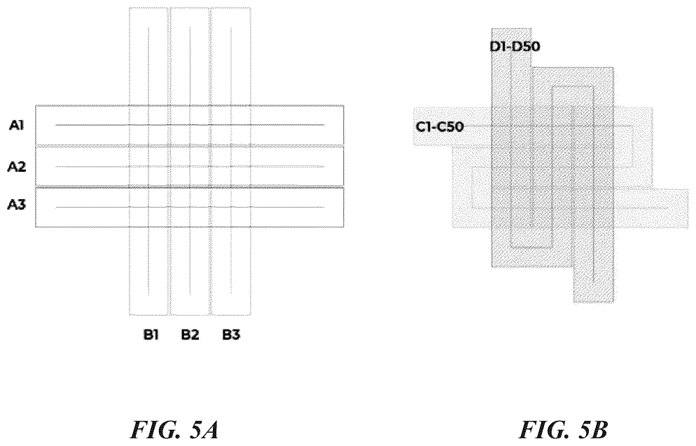

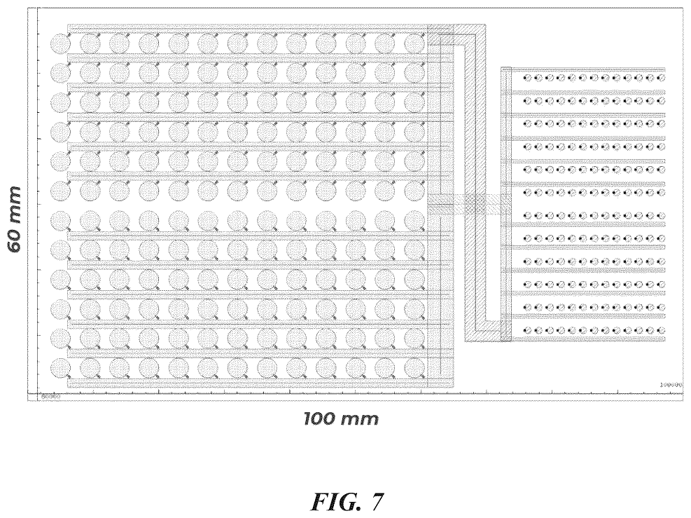

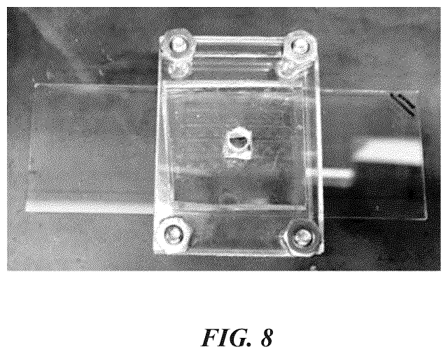

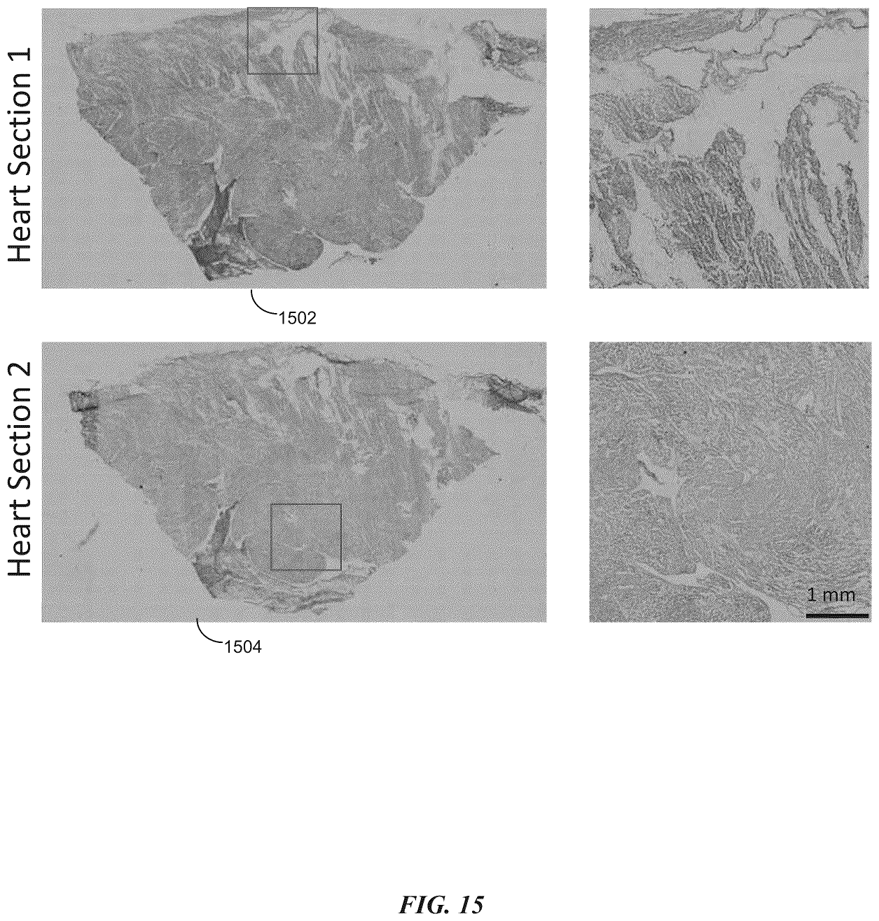

FIGS. 1, 2, 5A, 5B, 8, 9A, 9B, 10A, 10B, 11A, 11B, 11C, 11D, 12A, 12B, 13A, 13B, 13C, and 13D provide a description of exemplary devices for performing the methods described herein for spatially encoded biological assays. FIG. 3 illustrates an exemplary barcoding strategy in accordance with some embodiments. FIGS. 4 and 14 are flow diagrams illustrating methods for a spatially encoded biological assay in accordance with some embodiments. FIG. 6 compares footprints of different exemplary devices. FIG. 7 illustrates a 156×156 microfluidic device with dimensions of 10 cm×6 cm with a 3 mm×3 mm region of interest.

Although the following description uses terms “first,” “second,” “third,” etc. to describe various elements, these elements should not be limited by the terms. These terms are only used to distinguish one element from another. For example, a probe of a first type could be termed a probe of a second type, and, similarly, a probe of a second type could be termed a probe of a first type, without departing from the scope of the various described embodiments. The probe of a first type and the probe of a second type are both probes, but they are not the same probe.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used in the specification and claims, the term “molecular probe region” refers to a region of a probe that can be linked to a molecule in a sample.

As used in the specification and claims, the term “binder-tag conjugate” refers to a reagent that labels a molecule in a sample with a tag that identifies the molecule in the sample, and a moiety that can be linked to a probe as described in the methods provided herein, such as a probe of the probes of the first type.

As used in the specification and claims, the term “linked” or “linking” refers to direct and/or indirect linkages. A linkage can include a covalent linkage, or a noncovalent linkage, such as a hydrogen bond or ionic bond. Examples of linkages include antibody-antigen interactions, and nucleic acid hybridization.

As used in the specification and claims the term “nucleic acid” refers to at least two nucleotides covalently linked together. A nucleic acid generally will contain phosphodiester bonds, although in some cases nucleic acid analogs can be included, such as nucleic acid analogs that have alternative backbones such as phosphoramidite, phosphorodithioate, or methylphophoroamidite linkages; or peptide nucleic acid backbones and linkages. Other analog nucleic acids include those with bicyclic structures including locked nucleic acids, positive backbones, non-ionic backbones and non-ribose backbones. Modifications of the ribose-phosphate backbone may be done to increase the stability of the molecules; for example, PNA:DNA hybrids can exhibit higher stability in some environments.

As used in the specification and claims, the term “sequencing”, and the like refers to determination of information relating to the nucleotide base sequence of a nucleic acid or nucleic acid analog. Such information may include the identification or determination of partial as well as full sequence information of the nucleic acid. “Next generation sequencing” refers to sequence determination using methods that can determine many (typically thousands to billions) of nucleic acid sequences in a parallel manner, or alternatively using an ultra-high throughput serial process that itself can be parallelized.

As used in the specification and claims, the term “spatial barcode” refers to a molecular tag or moiety which a) displays a one-to-one relationship with a specific geometric region of a 2-D substrate, b) can be conjugated, linked, or otherwise chemically or physically bonded to a target analyte of interest, in such a way that following subsequent recovery and investigation of the molecular tag or moiety attached to the analyte, the analyte can be ascribed to having been located in said specific geometric region (in some embodiments, even in cases where the substrate has been dis-integrated and other means of obtaining spatial information have been lost), and c) permits identification of both the molecular tag and the analyte(s) attached to the tag, thereby providing a definite link between the tagged analyte and prior co-location with the specific geometric region identified by the molecular tag. A ‘set’ of spatial barcodes refers to the preponderance of such spatial barcodes that, taken together, span a geometric region of a substrate, such region being composed of smaller regions, each of which corresponding to exactly one of the spatial barcodes. Spatial barcodes include, but are not limited to, an oligonucleotide sequence or combination of sequences that can be conjugated to a plurality of binding sites of analytes in a tissue substrate (including, but not limited to, the polyadenylated tail of messenger RNA molecules), then subsequently read out by one or many of a variety of nucleotide sequencing modalities, including but not limited to next-generation sequencing or microscopy-based in situ sequencing techniques. Spatial barcodes may be delivered whole or in part (and subsequently combined with each other via, including but not limited to, ligation of nucleotide sequences) to specific geometric regions of a substrate in parallel, in series, or in a combination thereof by a set of microfluidics chips, or by a combination of a set of microfluidics chips and other device(s) designed to deliver reagents to specific geometric regions of a substrate, including, but not limited to, a device allowing for pooling of separate volumes of a set of reagents on top of a glass substrate in several noncontiguous regions corresponding to the locations of distinct tissue sections.

Techniques such as DBiT-seq can build barcodes specific to detector tixels (and hence to the spatial locations of detected molecules) by linking together two sets of reagents, with each unique combination of reagent referring to exactly one tixel. That is, each 2-D location can be represented by a 2-D barcode. Herein we describe a set of techniques in which the use of additional spatial barcodes (for example, a third set, a fourth set, or even further sets) can provide a third, fourth, or higher dimension to the barcode phase space. The additional barcodes can serve to distinguish the spatial locations of repeated combinations of lower-dimension barcodes from one another.

FIG. 4 illustrates an embodiment of a method in which a barcode structure can be generated. In this diagram, multiple sets of probes (N sets in total) are introduced to a sample, (e.g., an intact tissue section) in a spatially defined manner. Each set of probes can include nᵢ individually barcoded probes, where i=1, 2, . . . N. Thus for example a first set of probes could consist of molecules having a common part (e.g. an oligonucleotide sequence that binds to a specific sequence in a molecule in a sample (e.g. oligo(dT) binding to molecules with a poly(A) tail, or a ligation linking sequence intended to allow each set of barcodes to ligate to previous and subsequent sets), and spatial barcode encoding the probe's spatial delivery pattern, for example the number of its individual microchannel in a sequence of channels in a microfluidic chip.

The probes can be designed such that, they can be linked to one another. For example, one or more probe(s) can be linked in a manner that can permit identifying one or more spatial barcodes. In some embodiments, one or more probes can be linked in a manner which permits identifying the spatial barcodes of the linked probes. In some embodiments, the identity of the spatial barcodes is identified at the conclusion of the method. In some embodiments, the spatial barcodes include nucleic acid molecules, and are identified by nucleic acid sequencing. Thus, for example, in some embodiments, the first, second, and third spatial barcodes include nucleic acid molecules or nucleic acid analog molecules. In further embodiments identifying that the molecule is present in the sample can include sequencing nucleic acid molecules of the first, second, and third spatial barcodes. In some embodiments, sequencing can include next-generation sequencing, chain termination sequencing, or pyrosequencing. In some embodiments, sequencing is performed by next-generation sequencing. In some embodiments, sequencing includes producing sequencing constructs via tagmentation, and sequencing the sequencing constructs to produce the cDNA reads. Tagmentation refers to a modified transposition reaction, often used for library preparation, and involves a transposon cleaving and tagging double-stranded DNA with a universal overhang. Tagmentation methods are known.

In some embodiments, spatial barcodes can be identified using one or more techniques selected from mass spectroscopy (e.g., Maldi-T of, LC-MS/MS), nuclear magnetic resonance imaging, fluorescence imaging, and light detection.

In embodiments of methods provided herein, any technique known to one of skill in the art can be used to link probes. In some embodiments, probes are linked sequentially. For example, those of skill in the art are aware of exemplary techniques for linking nucleic acid molecules via one or more ligation(s). For example the SPLIT-seq technique (see, e.g., Rosenberg et al. (2018) Science 360:176-182) can produce cell-specific barcodes using a split/pool technique consisting of: 1) binding and synthesis of a first set of barcodes with reverse transcription (RT), 2) ligation of a second set of barcodes, 3) ligation of a third barcodes, and 4) PCR to hashtag single cells in microwells. Following PCR, the entire sequence of barcodes can be identified, for example by Next-Generation Sequencing.

Other exemplary techniques for linking probes include chemical linkage, such as, for example, Click Chemistry. Exemplary Click Chemistry techniques include, for example cycloaddition, azide-alkyne cycloaddition, azide-cyclooctyne cycloaddition, azide-dibenzocyclooctyne cycloaddition, copper(I)-catalyzed azide-alkyne cycloaddition, Diels-Alder reactions, inverse electron demand Diels-Alder reactions, inverse electron demand Diels-Alder reactions of a tetrazine and an olefin, inverse electron demand Diels-Alder reaction of a tetrazine and an triazine, inverse electron demand Diels-Alder reaction of a 1,2,4,5-tetrazine and an olefin, inverse electron demand Diels-Alder reaction of a 1,2,4,5-tetrazine and a 1,2,3-Triazine, inverse electron demand Diels-Alder Reaction of a trans-cyclooctene and a tetrazine, inverse electron demand Diels-Alder Reaction of a trans-cyclooctene and a 1,2,4,5-tetrazine, and photoclick reactions (e.g., 1,3-dipolar cycloaddition, tetrazole-alkene cycloaddition) (Mamidyala, Finn, Chem. Soc. Rev., 2010, 39, 1252-1261; also, Ramil, Lin, Photoclick chemistry: a fluorgenic light-triggered in vivo ligation reaction, Cur Opin Chem Biol, Volume 21, August 2014, Pages 89-95). In some examples, probes can be linked by photoactivation.

Attention is now directed toward embodiments of methods provided herein. FIG. 1 depicts one such embodiment in which a set of probes containing N barcodes (e.g., A1, A2, . . . AN), can be provided to N different regions. As depicted in FIG. 1, N=10, but N can be any positive integer, such as, for example, at least 5, at least 10, at least 20, at least 30, at least 40, at least 50, 5 to 500, 5 to 100, 10 to 90, 30 to 70, 40 to 60, or 50. This set of barcodes can intersect a second set of probes containing N barcodes (e.g., B1, B2, . . . BN) in two or more regions, (e.g., 201 and 202). Although as illustrated in FIG. 1, the number of “A” barcodes and the number of “B” barcodes can be the same, the number of “A” barcodes can be different from the number of “B” barcodes. There can be multiple (in this case, two, but there can be any number of intersections) spatial locations associated with a 2-D barcode AiBj, where i and j represent any integer between 1 and N. By introducing a third series of barcodes (e.g., C1 and C2), the multiple locations previously barcoded as AiBj can be uniquely barcoded locations (e.g., tixels). The barcode can include a 3-dimensional barcode denoted AiBjCk, where Ai and Bj are as before, and Ck=C1 or C2.

The probes can be provided, for example, by reagent deposition or by microfluidic flow. For example, FIG. 1 depicts embodiments in which probes containing barcodes A1, A2, . . . AN and probes containing barcodes B1, B2, . . . BN can be provided by microfluidic flow, and barcodes C1 and C2 can be provided by reagent deposition.

As depicted in FIG. 1, a plurality of probes of a first type can be provided to a first region 101 of a sample in accordance with some embodiments. At least a subset of the probes of the first type can include a first spatial barcode. At least a first probe of the probes of the first type can be linked to a molecule in the sample. A plurality of probes of a second type can be provided to a second region 102 of a sample. At least a subset of the probes of the second type can include a second spatial barcode different from the first spatial barcode. The first region 101 and the second region 102 can intersect at two or more noncontiguous locations (e.g., 103 and 104) on the sample. A first probe of the probes of the second type can be linked to the first probe of the probes of the first type. In some embodiments, a first probe of the probes of the second type can be linked to the first probe of the probes of the first type at an intersection 103 of the first region 101 and second region 102. In some embodiments, a second probe of the probes of the first type and a second probe of the probes of the second type can be linked at an intersection 104 of the first region 101 and second region 102. A probe of a third type can be provided to a third region (e.g., 105) of the sample. In some embodiments, the third region includes an intersection of the first and second regions. In some embodiments, the third region can be at least about 10-fold (e.g., at least about 50-fold, 100-fold, 150-fold, 200-fold, 300-fold, 400-fold, 500-fold, 600-fold, 700-fold, 800-fold, 900-fold, 1000-fold, 1500-fold, 2000-fold, 2500-fold, 3000-fold, 3500-fold, 4000-fold, 4500-fold, 5000-fold, 6000-fold, 7000-fold, 8000-fold, 9000-fold, or 10000-fold) larger than the area of an intersection of the first and second regions. A probe of the third type can include a third spatial barcode different from the first and second spatial barcodes. The probe of the third type can be linked to the first probe of the first type and/or the first probe of the second type. In some embodiments, the molecule is identified as present in the sample at an intersection based on at least the first, second, and third spatial barcodes. For example, a molecule can be identified as present at intersection 103 based on the first spatial barcode (e.g., A1), the second spatial barcode (e.g., B1), and a third spatial barcode (e.g., C1) of a probe of a third type provided to region 105 of the sample. A molecule can be identified as present at intersection 104 based on the first spatial barcode (e.g., A1), the second spatial barcode (e.g., B1), and a third spatial barcode (e.g., C2) of a probe of a third type provided to region 106 of the sample.

In some embodiments there can be more than two intersections between two sets of barcodes (e.g., Barcodes A and barcodes B). In some embodiments, the first and second regions can intersect at 2 or more, 3 or more 4 or more, 5 or more, 6 or more, 7 or more, 8 or more, 9 or more, 10 or more, 11 or more, 12 or more, 13 or more, 14 or more, 15 or more, 20 or more, 25 or more, 30 or more, or 35 or more noncontiguous locations on a sample. In some embodiments, a probe of the third type is provided to 2 or more, 3 or more 4 or more, 5 or more, 6 or more, 7 or more, 8 or more, 9 or more, 10 or more, 11 or more, 12 or more, 13 or more, 14 or more, 15 or more, 20 or more, 25 or more, 30 or more, or 35 or more intersections of the first and second regions.

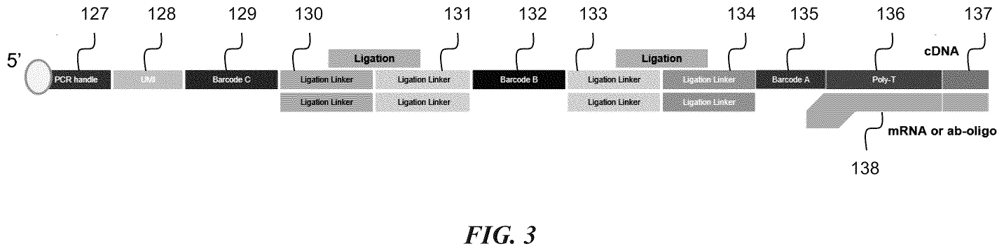

For example, FIG. 2 depicts an embodiment in which a set of Barcodes A intersect a set of Barcodes B in twelve noncontiguous locations 201, 202, 203, 204, 205, 206, 207, 208, 209, 210, 211, and 212. In an intersection, a tixel can be barcoded as AiBjCk, where i and j are each an integer between 1 and N, and k=1, 2, . . . 12. Thus, for example, the first region 101 and the second region 102 can intersect at twelve noncontiguous locations 103, 104, 107, 108, 109, 110, 111, 112, 113, 114, 115 and 116. Thus, for example, a molecule can be identified at intersection 103, 104, 107, 108, 109, 110, 111, 112, 113, 114, 115 or 116 based on spatial barcode A1 of a probe provided to region 101, spatial barcode B2 of a probe provided to region 102, and a spatial barcode C of a probe provided to region 105, 106, 117, 118, 119, 120, 121, 122, 123, 124, 125, or 126, respectively, of a sample.

In some embodiments, additional types of probes can be provided to the sample. For example, a probe of a fourth type (or a plurality thereof) can be provided to a fourth region (e.g., a fourth region comprising the first intersection of the first and second regions) of the sample, wherein the probe of the fourth type can include a fourth spatial barcode different from the first, second and third spatial barcodes. The probe of the fourth type can be linked to one or more of the first probe of the first type, first probe of the second type, and probe of the third type. In further embodiments, the molecule can be identified as present in the sample at the first intersection of the first and second regions based on at least the first, second, third and fourth spatial barcodes. In further embodiments, the molecule can be identified as present at an intersection of the first, second, third and fourth regions.

FIG. 3 representationally depicts an exemplary probe of a first type linked to a molecule (e.g., nucleic acid, mRNA a binder-tag conjugate, a protein-nucleic acid tag, an antibody-oligonucleotide conjugate, or an antibody-DNA tag (ADT)), a probe of a second type linked to the probe of a first type, and a probe of a third type linked to the probe of a second type. In embodiments in which the molecule is a binder-tag conjugate, a protein-nucleic acid tag, an antibody-oligonucleotide conjugate, or an antibody-DNA tag (ADT), the method can include providing the binder-tag conjugate, protein-nucleic acid tag, antibody-oligonucleotide conjugate, or antibody-DNA tag (ADT) to the sample. In some embodiments, the probe of a first type can include a first spatial barcode 135 (e.g., “Barcode A”), a ligation linker 134, and a first molecular probe region 136 that is linked to the molecule. For example, the first molecular probe region can include a first molecular probe region 136 that is oligo(dT). In further embodiments, the molecule can include a polyadenylated region 138, for example as described in [(Proudfoot, Genes Dev. 2011 Sep. 1; 25(17):1770-82. doi: 10.1101/gad.17268411)]. cDNA optionally can be prepared, for example by reverse transcription (RT) to the molecule. For example, as illustrated in FIG. 3, cDNA 137 can be prepared to the molecule. The molecule can be, for example, a nucleic acid, such as mRNA (e.g., single-stranded mRNA), or a single-stranded or double-stranded DNA barcode conjugated to a protein-specific antibody. The probe of a second type can include a second spatial barcode 132 (e.g., “Barcode B”) different from the first spatial barcode, a ligation linker 133 for linking to the probe of the first type, and a ligation linker 131 for linking to a probe of the third type. The probe of a third type can include a third spatial barcode 129 (e.g., “Barcode C”) different from the first and second spatial barcodes, and a ligation linker 130 for linking to the probe of the second type. In some embodiments, a probe of a first type and probe of a second type can be linked, for example, by ligation. The probe of a third type can be linked to a probe of the second type by ligation. Ligation techniques are described, for example, in (Rosenberg et al, Single-cell profiling of the developing mouse brain and spinal cord with split-pool barcoding, Science:13 Apr. 2018: 176-182). In some examples, a universal ligation linker sequence includes a sequence complementary to the ligation linkers of two probes to be linked, wherein the universal ligation linker can include a sequence complementary to the ligation linker sequence of probes to be linked. The length of a universal ligation linker can vary. For example, a universal ligation linker can have a length of 10 to 100 nucleotides (e.g., 10 to 90, 10 to 80, 10 to 70, 10 to 60, 10 to 50, 10 to 40, 10 to 30, 10 to 20, 20 to 100, 20 to 90, 20 to 80, 20 to 70, 20 to 60, 20 to 50, 20 to 40, or 20 to 30 nucleotides). In some embodiments, a universal ligation linker can have a length of 10, 15, 20, 25, 30, 35, 40, 45, 50, 55, 60, 65, 70, 75, 80, 85, 90, 95, or 100 nucleotides. Longer universal ligation linkers are contemplated herein. A universal ligation linker can be annealed to a ligation linker of a probe provided to a sample. In some examples, a universal ligation linker can be separately provided to a sample. A probe of a third type can be linked to a probe of a first type and/or a probe of a second type.

One or more of the probe of a first type, probe of a second type, and probe of a third type can include a Unique Molecular Identifier (UMI) 128, which can distinguish PCR amplicon copies from one another. For example, the UMI can distinguish PCR amplicon copies in a prepared library, for example as described in Liu et al. (2020). High-Spatial-Resolution Multi-Omics Atlas Sequencing of Mouse Embryos via Deterministic Barcoding in Tissue. doi: 10.1101/788992. One or more of the probe of a first type, probe of a second type, and probe of a third type can include a PCR handle 127. For example, the probe of a third type can include a PCR handle 127. In some examples, the PCR handle 127 is at the 5′ end of the probe of a third type. In some embodiments, the PCR handle can be terminally functionalized with biotin. In some embodiments, a second PCR handle can be added (e.g., at the opposite end of PCR handle 127) by template switching. Template-switching (also known as template-switching polymerase chain reaction (TS-PCR)) is a method of reverse transcription and polymerase chain reaction (PCR) amplification that relies on a natural PCR primer sequence at a polyadenylation site, also known as the poly(A) tail, and adds a second primer, such as through the activity of murine leukemia virus reverse transcriptase (see, e.g., Petalidis L. et al. Nucleic Acids Research. 2003; 31 (22): e142). It is understood that the probes can be provided and/or linked in any order. For example, an order can be selected from among ABC, BAC, CAB, ACB, BCA, and CBA.

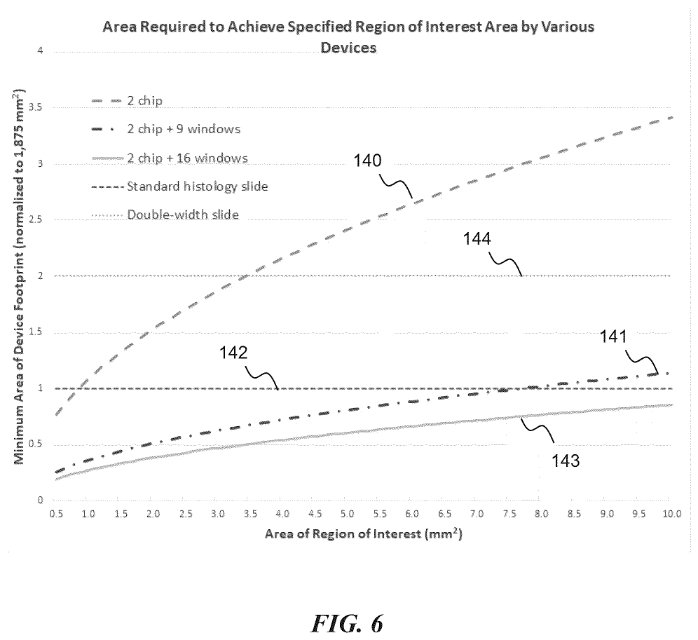

In still other embodiments, tixels can be barcoded by other combinations of any number of series of barcodes. For example, FIG. 5A and FIG. 5B depict a set of probes of a first type, a set of probes of a second type, a set of probes of a third type, and a set of probes of a fourth type. In some embodiments, probes can be delivered via a total of four microfluidic chips. In an exemplary embodiment as illustrated in FIG. 5A, Probes containing barcodes A1, A2, A3 and B1, B2, B3 can flow at right angles to one another utilizing, for example, channels with width of one millimeter, defining a 3×3 grid of 1×1 millimeter regions of interest. Probes containing barcodes C1, C2, . . . C50 and D1, D2, . . . D50 can flow in a mutually re-intersecting pattern, such that the nine intersection areas of barcodes A1, A2, A3 and B1, B2, B3 contain one group of the 2,500 unique combinations of C and D barcodes. It is understood that the probes can be provided in any order, for example, the probes can be provided in an order selected from among ABCD, BACD, CABD, ACBD, BCAD, CBAD, CBDA, BCDA, DCBA, CDBA, BDCA, DBCA, DACB, ADCB, CDAB, DCAB, ACDB, CADB, BADC, ABDC, DBAC, BDAC, ADBC, and DABC.

Thus in this embodiment, a location (e.g., a tixel) can have a 4-D barcode, AiBjCkDl, where i,j=1, 2, 3, and k,1=1, 2, . . . 50. To illustrate the super-linear scaling of tixel count with channel count featured this method, note that, in this embodiment, the total number of locations (e.g., tixels) is (i)(j)(k)(1)=9×2,500=22,500, and can cover an area of, for example, 3×3 millimeters=9 square millimeters (in the case of 10 micron channels). The Barcode delivery chips can use only 106 individual barcodes (3+3+50+50) and four chips of normal footprint (e.g. 75 mm×25 mm, the most common size for glass slides on which tissue is typically mounted). To achieve the same number of locations (e.g., tixels) with only two series of barcodes can require 150 input channels on each chip, for a total of 300 barcodes, and each chip can be significantly larger (e.g., approximately 10 cm×6 cm) to accommodate inlet and outlet ports at a standard inlet well spacing (see, e.g., FIG. 7).

FIG. 7 depicts a chip for a two-chip technique that can achieve a 3 mm×3 mm region of interest. The chip occupies a very large (by microscopy standards) footprint of 10 cm×6 cm, and has 156 inlet and outlet wells. To further increase the region of interest can require the addition of even more inlet and outlet wells, which can further increase liquid handling complexity and further amplify the chip footprint, which, as illustrated in FIG. 7 can already approach the limits of soft lithographic fabrication and execution capabilities.

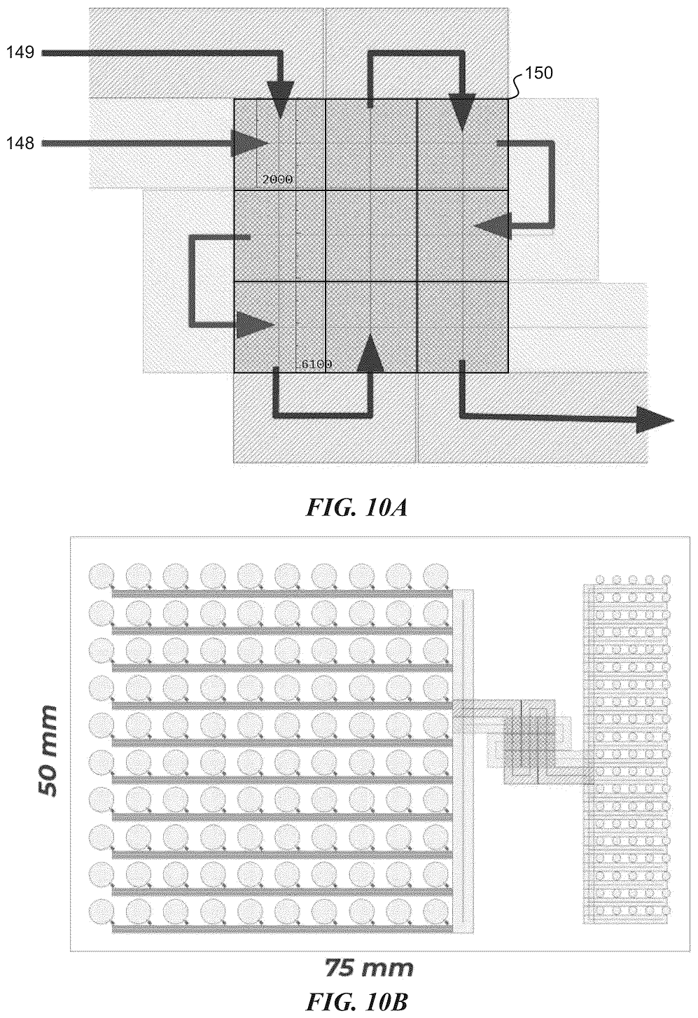

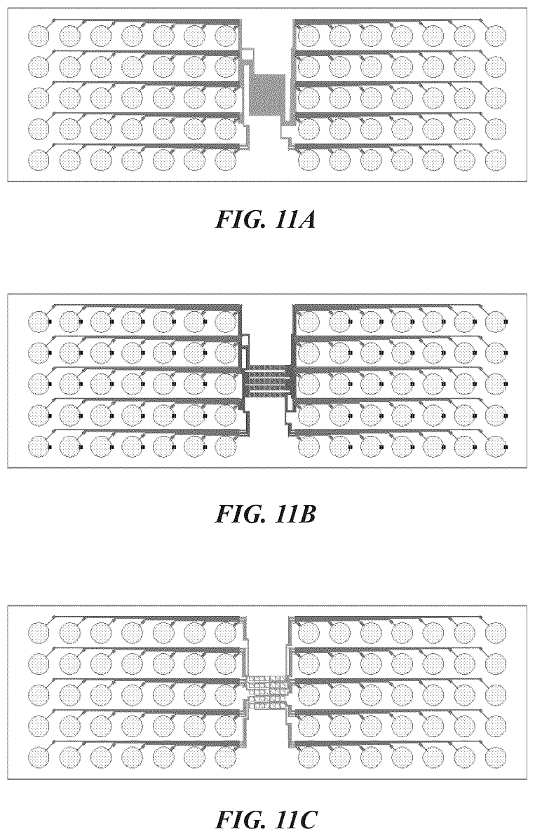

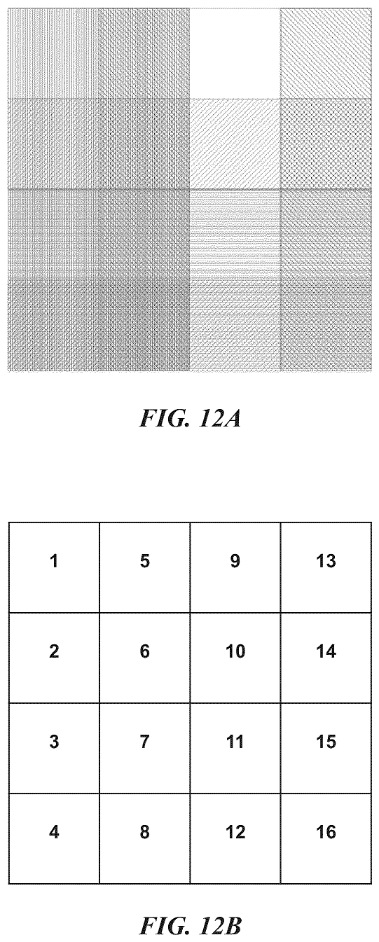

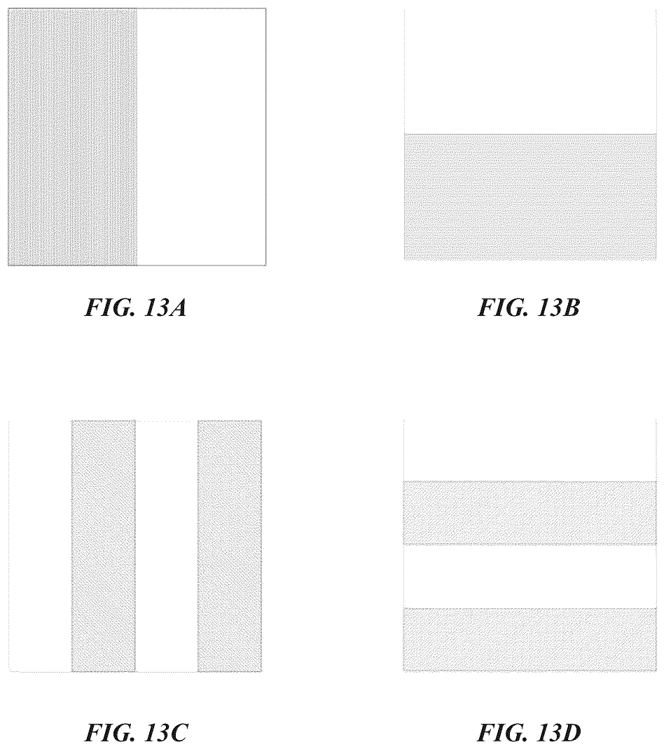

FIG. 6 illustrates a difference in scaling between a two-chip technique and exemplary methods as described herein in which three or more probes are provided. Each curve corresponds to two or more microfluidic chips with microchannels 10 microns wide. Curve 140 (large dashes) corresponds to a two-chip technique (e.g., FIG. 7), such as outlined in Liu et al. (2020). High-Spatial-Resolution Multi-Omics Atlas Sequencing of Mouse Embryos via Deterministic Barcoding in Tissue. doi: 10.1101/788992. Curve 140 illustrates that, to achieve larger regions of interest, devices with rapidly growing footprints can be required. Technical difficulty can scale linearly with the area of a device (e.g., execution risk, and fabrication complexity). Methods provided herein can achieve larger regions of interest with less technical difficulty compared to two-chip techniques. For example a device for use in methods provided herein can include three chips, such as, for example, two chips with crossflowing 10 micron microfluidic channels and a third chip with 9 reagent deposition windows (e.g., curve 141, dash-dots), for example as illustrated in FIGS. 9A and 9B, and in FIGS. 10A and 10B) can achieve regions of interest of over 7 mm² before requiring a footprint as large as a standard glass slide used commonly in histology to mount tissue sections (area indicated as line 142). Curve 143 (solid line) illustrates that an analogous device with 16 discrete location (e.g., tixel) groupings (for example, via a third chip with 16 windows, or additional chips defining a 4×4 grid such as that described in FIGS. 12A-B, 13A, 13B, 13C and 13D, and Table 1) can perform even better, and can achieve regions of interest of more than 10 mm² without exceeding the area of a standard histology slide. Line 144 indicates the area of a double-width histology slide.

The curves in FIG. 6 were generated as follows. Line 140 (dashed) corresponds to a 10 micron DBiT-seq device using two chips on a 25×75 mm footprint with 50 inlet wells (and microfluidic channels) on each chip. Each microchannel is 10 microns wide and is separated from the neighboring channel by 10 microns (e.g., 10 microns of solid PDMS). Thus, 50 such channels can occupy a linear dimension of 10×2×50=1000 microns, or 1 mm. The region of interest (e.g., an area of the sample, such as, for example, tissue, over which the first two flow stages intersect) is 1 mm×1 mm=1 mm². By inverting this formula, from the region of interest area A square millimeters the number of microfluidic channels N of width w microns can be calculated using the following Equation 1:

$$N = ( 1000 ) {A}^{1 / 2} / ( 2 w ) \qquad ( Equation 1 )$$

For a DBiT-seq device, an overlap area of N×N tixels can require N input channels on a microfluidic chip. To maintain a standard 384-well plate spacing of 4.5 mm between inlet wells, an average of 20.25 square millimeters can be allocated to each inlet well. If the total area of the device allocated to inlet wells is not much more than approximately half of the device (to allow space for the active area of the chip, as well as outlet wells), one can estimate the required footprint of such a device F square millimeters using the following Equation 2:

$$F = 2 N \times {4.5}^{2} / S \qquad ( Equation 2 )$$

wherein:
area S=25 mm×75 mm=1,875 mm² is divided into F in order to normalize F against the area of a standard histology slide, commonly used to mount sectioned tissue samples; and
F, the footprint of a DBiT-seq style device to achieve a given area of region of interest, thus estimated and normalized, is plotted in FIG. 6 as dashed curve 140.

A similar strategy can calculate the dashed-dotted curve 141, for a device using: 1) two chips with crossflowing microfluidic channels in a mutually re-intersecting pattern, multiplexed 9 times; and 2) nine reagent deposition windows (e.g., as described in FIGS. 9A and 9B); or two chips using crossflowing microfluidic channels that form, for example, a 3×3 grid (e.g., FIGS. 5A and 5B), except that N is computed differently. In this case, the first two flow stages do not need N inlet wells. Rather, since they are multiplexed 9 times (e.g., by the third chip or by crossflowing microfluidic channels that form, for example, a 3×3 grid, only N/9 inlet wells are needed on the first two flow stages. Therefore in this case N can be calculated using the following Equation 3:

$$N = ( 1000 ) {( A / 9 )}^{1 / 2} / ( 2 w ) , \qquad ( Equation 3 )$$

and F, the footprint of a device described in FIGS. 5A and 5B, and FIGS. 9A and 9B, to achieve a given area of region of interest, thus estimated and normalized, was calculated as previously indicated, and plotted as dashed-dotted curve 141.

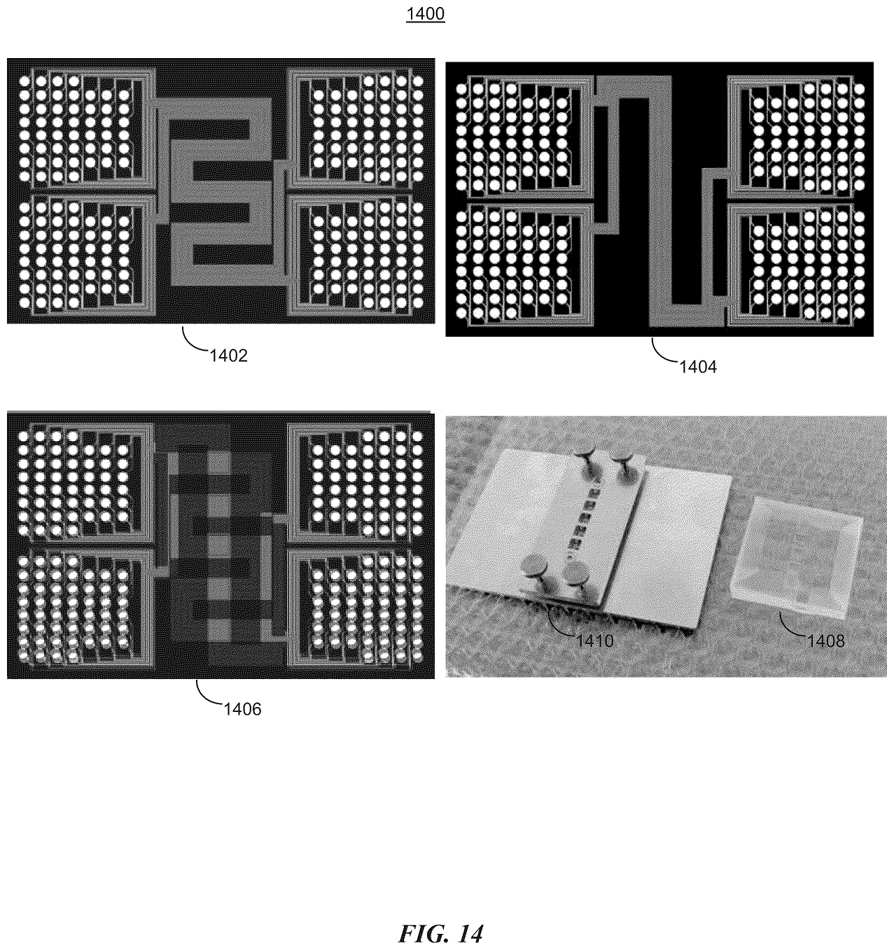

Similarly in an embodiment in which one or more microfluidic chips or photomasks is used to multiplex the intersection areas of the first two flow stages (see FIGS. 12A and 12B and FIGS. 13A, 13B, 13C, and 13D, for example) 16 times, N can be calculated using the following Equation 4:

$$N = ( 1000 ) {( A / 16 )}^{1 / 2} / ( 2 w ) . \qquad ( Equation 4 )$$

F, the footprint of a device described in FIGS. 12A and 12B and FIGS. 13A, 13B, 13C, and 13D, to achieve a given area of region of interest, thus estimated and normalized, can be calculated as in Equation 2, and is plotted as the solid curve 143.

More generally when the intersection areas of the first two flow stages are multiplexed M times by the following flow stages or other processes, N can be calculated by the following Equation 5:

$$N = ( 1000 ) {( A / M )}^{1 / 2} / ( 2 w ) , \qquad ( Equation 5 )$$

and the footprint, F, can be calculated by the following Equation 6:

$$F = 2 ( 1000 ) ( 4 . {5}^{2} ) ( 1 / S ) {( A / M )}^{1 / 2} / ( 2 w ) . \qquad ( Equation 6 )$$

M being greater than unity is the source of super-linear scaling or region of interest area with device footprint underpinning this invention.

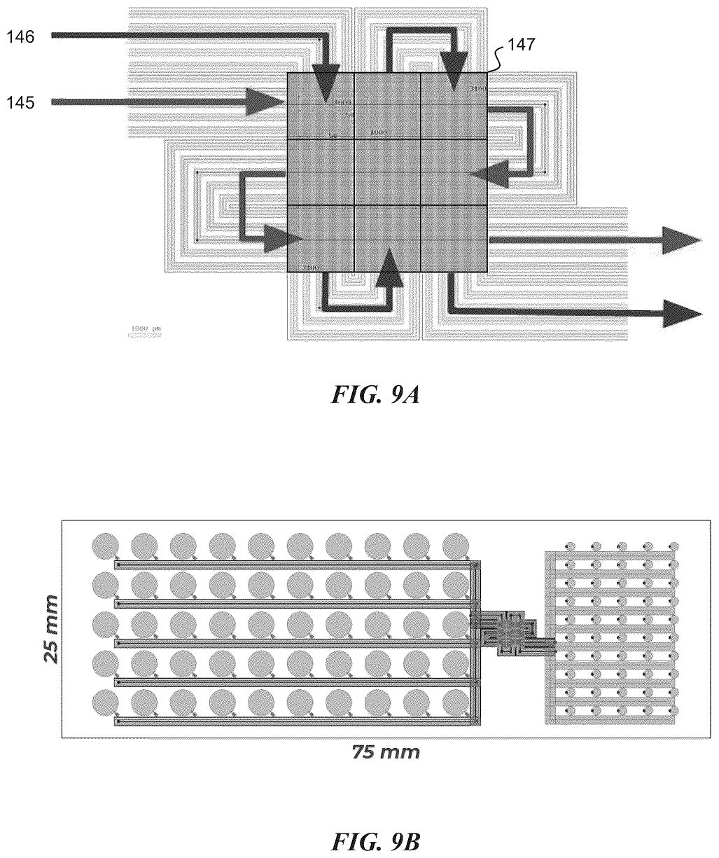

In some examples, the probes of the first type and the probes of the second type can be provided by microfluidic flow, and the probe(s) of the third type can be provided by reagent deposition. In some embodiments, sets of barcodes can be provided to a sample (e.g., a tissue) utilizing wells which include voids in an otherwise solid gasket, into which barcoded reagents can be delivered directly (e.g., via pipette or other liquid handling system, automated or otherwise). FIG. 8 shows an example of such a device with only one window. It is understood that a device for use in methods provided herein can contain any number of windows, such as for example, 1 or more, 4 or more, 9 or more, 16 or more, 25 or more, 36 or more, 49 or more, 64 or more, 81 or more, 100 or more, 121 or more, or 144 or more windows, such as 1, 2, 3, 4, 5, 6, 7, 8, 9, 10, 11, 12, 13, 14, 15, 16, 17, 18, 19, 20, 25, 36, 49, 64, 81, 100, 121, or 144 windows. In some examples, the windows can be arranged in an N×N grid, in which N is an integer from 1-100, such as 1-20, 1-10, 11-20, 1, 2, 3, 4, 5, 6, 7, 8, 9, 10, 11 or 12. For example, the windows can be arranged in a 2×2, 3×3, 4×4, 5×5, 6×6, 7×7, 8×8, 9×9, 10×10, 11×11, or 12×2 grid. In some examples, the windows can be arranged in an N×M grid, in which N and M are independently an integer from 1-100, such as 1-20, 1-10, 11-20, 1, 2, 3, 4, 5, 6, 7, 8, 9, 10, 11 or 12. For example, the windows can be arranged in a 1×2, 1×3, 1×4, 1×5, 1×6, 1×7, 2×3, 2×4, 2×5, 2×6, 2×7, 3×4, 3×5, 3×6, 3×7, 4×5, 4×6, 4×7, 5×6, 5×7, or 6×7 grid.

FIGS. 9A-9B depict one embodiment that utilizes this technique to amplify the region of interest of a microfluidics-based reagent delivery system. FIG. 9A shows an exemplary flow pattern of two microfluidics chips with microfluidic channels of a constant width (e.g., 10 microns). As illustrated in FIG. 9A, a first group of channels 145 weaves left to right, then right to left, then left to right. A second group of channels 146 weaves top to bottom, then bottom to up, then top to bottom. By virtue of their intertwining pattern, a 3×3 grid of tixel groups 147 is generated. Each tixel grouping can be an N×N grid of tixels covering a 1×1 mm region of tissue, for example as described in Liu et al. (2020). High-Spatial-Resolution Multi-Omics Atlas Sequencing of Mouse Embryos via Deterministic Barcoding in Tissue. doi: 10.1101/788992, with each tixel barcoded AiBj, with, for example i,j=1 . . . 50. Each square window in the 3×3 grid of tixel groups 147 can correspond to an open well in a third microfluidic chip (for example a gasket) which can guide the delivery of nine additional barcodes C1, . . . C9. For example, each window can be 1 mm×1 mm in size, with 50 micron gaps between windows. This can be achieved, for example, by materials such as acrylic or other plastics, or PDMS or other elastomeric resins affixed to a stiff backing. The device can have a 9 mm² region of interest and a 75 mm×25 mm footprint, which can correspond to the footprint of a standard histology slide (FIG. 9B).

In embodiments in which a 50×50 grid of tixels is repeated 9 times, a combination of three chips (including a total of 109 barcodes instead of 100, or a factor of 1.09×) can generate 50×50×9 tixels, or 22,500 tixels. The region of interest linear dimension can increase from 1 mm to 3 mm (increased by a factor of 3×), and the overall area of sample (e.g., tissue) assayed can be multiplied by a factor of 9×. Thus, super-linear scaling can be achieved. Since individual tixels can be 10 microns by 10 microns, and spaced at 20 microns center-center, single-cell resolution can be maintained. It will also be noted that inlet wells (FIG. 9B, left side) can maintain 4.5 mm center-center separation, which adheres to the industry standard for 384-well PCR plates, assisting with economic scalability. Outlet wells do not necessarily reflect this spacing, but outlet wells can be addressed by a common vacuum gasket without the need for individual well addressing. Thus, standard spacing of outlet wells is not necessary.

FIGS. 10A and 10B depict an embodiment consisting of three microfluidic chips, wherein the first and second microfluidic chips have 100 inlet wells (FIG. 10B). FIG. 10A shows an exemplary flow pattern in which a first group of channels 148 weaves left to right, then right to left, then left to right. A second group of channels 149 weaves top to bottom, then bottom to top, then top to bottom. By virtue of their intertwining pattern, a 3×3 grid of tixel groups 150 is generated. A third chip contains nine individual windows, each 2×2 mm in size with 50 μm spacing. Thus 100+100+9=209 barcodes are included. The region of interest dimension increases from 2 mm to 6 mm, and the area increases from 4 mm² to 36 mm². As described above, this represents super-linear scaling of a ROI dimension and achieves single-cell spatial resolution.

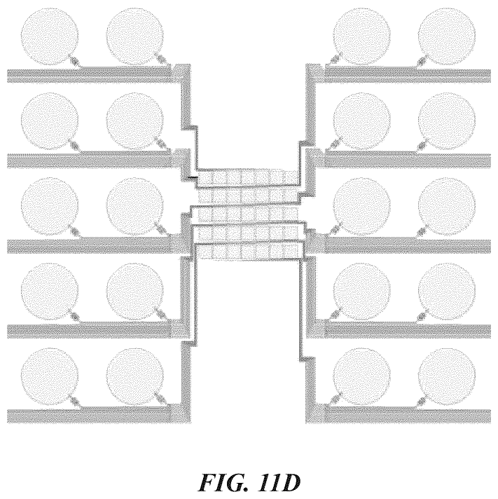

FIGS. 11A-11D depict an embodiment in which one or more barcode series in addition to the first two (FIGS. 11A and 11B) can be delivered via microfluidic channels. Microfluidic channels fed by inlet wells (FIGS. 11C and 11D) can deliver a third series of barcodes to a 5×7 grid of regions of interest. This method can include larger separation between windows that deliver the third barcode set, thereby increasing the center-center distance of some of the tixels.

Thus, in some embodiments of the methods provided herein, the probes are provided by microfluidic flow. For example, the probes of the first type, the probes of the second type, and the probe(s) of the third type can be provided by microfluidic flow. In further embodiments, microfluidic flow of a probe of the third type and/or fourth type can be at least about 2-fold (e.g., at least about 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 60-fold, 70-fold, 80-fold, 90-fold, 100-fold, 150-fold, 200-fold, 300-fold) wider than the width of the microfluidic flow of the probes of the first type and the probes of the second type

In some embodiments, a probe can include a photoactivateable group, such as, for example, a photo-activateable oligonucleotide(s) (see e.g. GeoMX DSP (Merritt, C. R., Ong, G. T., Church, S. E. et al. Multiplex digital spatial profiling of proteins and RNA in fixed tissue. Nat Biotechnol 38, 586-599 (2020), doi.org/10.1038/s41587-020-0472-9) or Transcriptome in vivo Analysis (TIVA) (Lovatt et al, Transcriptome in vivo analysis (TIVA) of spatially defined single cells in live tissue, 2014, Nature Methods 11:190-196). In further embodiments, providing a probe to a region of a sample can include photoactivation and/or photoinactivation (see e.g. van der Kemp, P. A., Blais, J.-C., Bazin, M., Boiteux, S. and Santus, R. (2002), Ultraviolet-B-induced Inactivation of Human OGG1, the Repair Enzyme for Removal of 8-Oxoguanine in DNA. Photochemistry and Photobiology, 76: 640-648. doi:10.1562/0031-8655(2002)0760640UBIIOH2.0.CO2 and Defrancq et al, WO 2008/113686 A2). For example, a photoactivateable group can be preferentially activated and/or inactivated at sub-region of a sample (e.g., a tissue) utilizing photomasks and/or collimated illumination devices. In some embodiments, linking two or more probes can include photoactivation and/or photoinactivation.

In some embodiments, a sample (e.g., a tissue) can be successively bifurcated For example, a sample can be bifurcated by photoactivation and/or microfluidic-based delivery. Bifurcation can double the number of regions of interest by an on/off pattern in a barcode sequence. The number of bifurcations can be increased, for example, by one or both of: a) a bifurcation substrate (e.g. microfluidic chip or photomask) with a high spatial fidelity; and b) a high barcode-recording fidelity of the medium in which the on/off barcode is recorded, e.g. an oligonucleotide sequence formed by repeated ligation, or selectively adapted by photo-cleaving at pre-determined sites.

Bifurcating a sample (e.g., a tissue) with binary on/off patterns can hashtag sub-regions with unique barcodes. An on/off pattern can be imposed employing one or more of various methods, including a chemical sequence deposited by reagent delivery, or a photomask selectively blocking portions of an incident collimated light source, e.g. a UV illuminator as is used commonly in soft lithographic methods.

FIGS. 12A-12B and 13A-13D depict an embodiment in which four patterns (FIGS. 13A-13D), such as four masks, can be combined to define a 4×4 grid of 16 sub-regions (FIG. 12A), each displaying a unique pattern of on or off for the four masks. For example, as illustrated in FIG. 12B and Table 1, the upper left corner 1 can be characterized by the sequence, “On, off, off, off.” The lower right corner 16 can be characterized by “Off, on, on, on.” In the case of N=4, Table 1 details an exemplary numbering scheme and spatial barcodes for 16 spatially encoded regions (FIGS. 12A and 12B).

One embodiment for defining hashtags encoding spatial locations of tixel groupings is illustrated in FIGS. 12A-B can utilize the photomasks shown in FIGS. 13A, 13B, 13C, and 13D. Each column pertains to one region as described in Table 1. A “1” in an entry in Table 1 indicates that the given barcode has been activated in that region (e.g., by photoactivation achieved by a mask that permits light through in that spatial region), while a “0” indicates that a barcode is not activated (e.g., a particular mask blocks light in that region, and even though barcode has been delivered to that region, it has not been activated). By sequentially barcoding regions (e.g., by applying photomasks represented by each column to selectively activate photo-activatable barcode elements), the hashtag shown in the right-most column can be generated on a sample (e.g., a tissue sample), thereby encoding target(s) in a spatial region of a sample (e.g., a tissue) with an identifiable barcode (e.g., identifiable in post-processing, such as, for example, by Next-Generation Sequencing).

TABLE 1

| | Masks | | | | |
|---|---|---|---|---|---|
| Region | A | B | C | D | Hashtag |
| 1 | 1 | 0 | 0 | 0 | 1000 |
| 2 | 1 | 0 | 0 | 1 | 1001 |
| 3 | 1 | 1 | 0 | 0 | 1100 |
| 4 | 1 | 1 | 0 | 1 | 1101 |
| 5 | 1 | 0 | 1 | 0 | 1010 |
| 6 | 1 | 0 | 1 | 1 | 1011 |
| 7 | 1 | 1 | 1 | 0 | 1110 |
| 8 | 1 | 1 | 1 | 1 | 1111 |
| 9 | 0 | 0 | 0 | 0 | 0000 |
| 10 | 0 | 0 | 0 | 1 | 0001 |
| 11 | 0 | 1 | 0 | 0 | 0100 |
| 12 | 0 | 1 | 0 | 1 | 0101 |
| 13 | 0 | 0 | 1 | 0 | 0010 |
| 14 | 0 | 0 | 1 | 1 | 0011 |
| 15 | 0 | 1 | 1 | 0 | 0110 |
| 16 | 0 | 1 | 1 | 1 | 0111 |

In general, given N patterns, there can be 2^{N} possible combinations.

In some embodiments, the methods include imaging the sample to produce a sample image. The imaging, in some embodiments, can be with an optical or fluorescence microscope.

In some embodiments, identifying that a molecule is present in the sample at a location can include in silico reconstruction of a target location map. The reconstructed target location map can be correlated to a sample image to identify the spatial location of individual tixels.

Also provided herein are compositions for use in methods provided herein, intermediate compositions produced during methods provided herein. For example, provided herein are compositions containing two or more microfluidic chips. In some embodiments, when positioned at the same location, microfluidic channels of one chip crossflow and intersect with microfluidic channels of another chip in two or more locations. Also provided herein are compositions further containing a chip that includes two or more reagent deposition windows. In some embodiments, the reagent deposition windows are located at one or more positions corresponding to intersections of microfluidic channels between two microfluidic chips. In some embodiments, the compositions provided herein include a probe of a first type, or a plurality thereof, a probe of a second type, or a plurality thereof, and a probe of a third type, or a plurality thereof.

In some embodiments, compositions provided herein include a biological sample. In further embodiments, the biological sample is attached to a tissue slide. In some embodiments, the biological sample includes a molecule as described herein, such as mRNA. In further embodiments, the mRNA includes a poly A tail. In some embodiments, compositions provided herein include a binder-tag conjugate.

In some embodiments, compositions provided herein include a conjugate described herein. In some embodiments, the conjugate includes a first, second, and third spatial barcode. In some embodiments, the conjugate includes one or more PCR handle sequence(s), a universal molecular identifier (UMI) sequence, and one or more ligation linker sequence(s).

Also provided are kits containing one or more compositions provided herein. In some examples, the kits can contain one or more regents selected from among tissue fixation reagents, reverse transcription reagents, ligation reagents, polymerase chain reaction reagents, template switching reagents, and sequencing reagents.

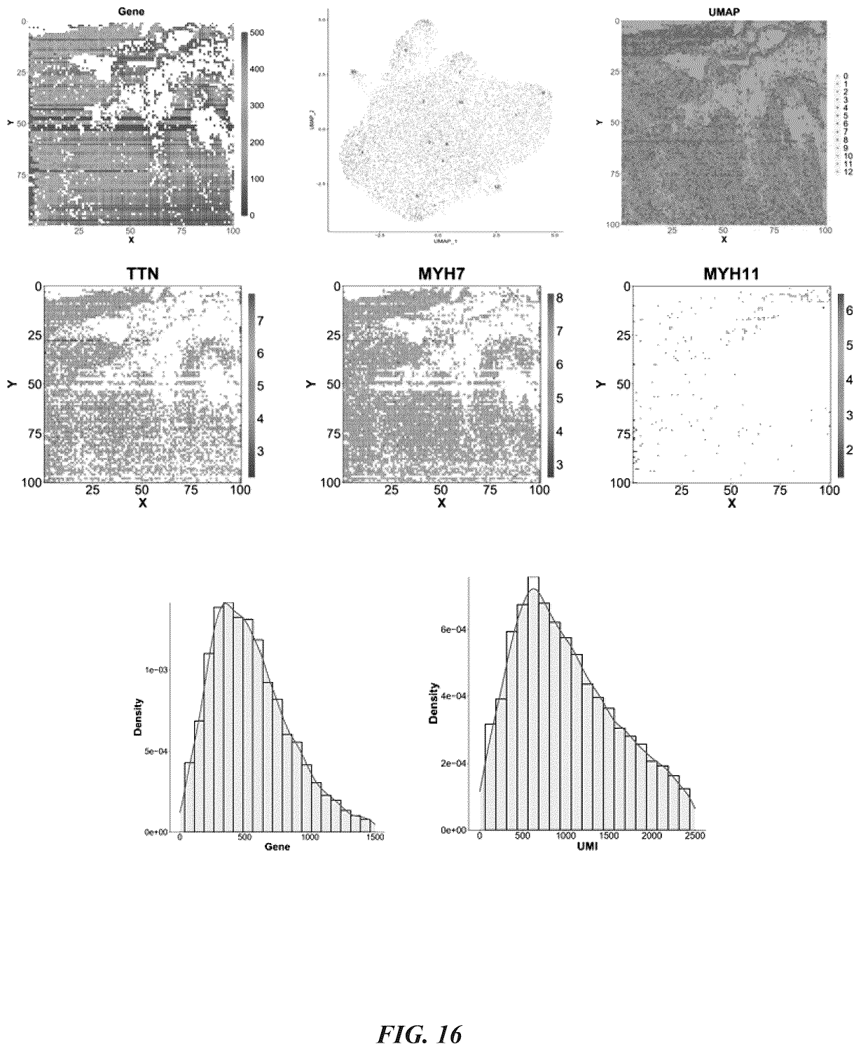

FIG. 14 depict a device 1400 producing 5 discrete regions of interest, either on the same tissue section (e.g., a tissue section as discussed with reference to FIG. 15), or on 2, 3, 4 or 5 separate tissue sections, as described below.

Device 1400 includes three chips constructed according to the following specifications. First chip 1402 has 100 disparate inlets, outlets, and channels with width ranging from 100 um near the ports to 20 um in the region of interest. Second chip 1404 has channels similar in dimensions to the first chip. The 100-channel manifold of second chip 1404 runs along a single vertical strip, and therefore each of the channels of second chip 1404 intersect with each of the channels of first chip 1402 in 5 locations (one intersection for each ROI). When applied in series to a substrate as shown in schematic 1406, it can be readily perceived when observing schematic 1406 that the channel manifolds will intersect in 5 distinct regions of interest, on 1, 2, 3, 4, or 5 tissue sections, therefore generating 5 repeats of each combination of the first two spatial barcodes. Third chip 1408 has 5 open windows corresponding to each of the 5 ROIs. In some embodiments, the material between the windows has a substantially flat lower surface so that reagent or lysis buffer loaded into each of the windows does not leak between ROIs, which would cause erroneous spatial reconstructions in downstream analysis. Third chip 1408 permits application of a third set of 5 barcodes to the 5 ROIs, or equivalently, physically-separated tissue dissociation and cellular/nuclear lysis of the 5 ROIs. In some embodiments, care is taken when applying third chip 1408 so that the 5 open windows overlap with the 5 areas of intersection between first chip 1402 and second chip 1404.

In some embodiments, first chip 1402, second chip 1404, and third chip 1408 are strongly clamped to the sample substrate. In some embodiments, such clamping is achieved using clamp 1410, which is a specialized, sturdy clamp capable of applying uniform sealing pressure across the entire active area of device 1400 in order to attain sufficient simultaneous sealing pressure across each of the 5 ROI's.

Since first chip 1402 and second chip 1404 each have 100 channels, and each channel has width of 20 um, with 20 um spacing between channels, each ROI has 4×4 mm² area. Since there are 5 ROI's, this yields 5×4×4=80 square millimeters of assay area on a single device. Compared, for example, to a set of chips producing one ROI with 6.25 square millimeters, this doubles the chip area (from 3 square inches to 6 square inches) but multiplies the active area by 80/6.25=12.8 times.

FIG. 15 depicts images of two separate fresh-frozen human heart tissue sections, section 1502 and 1504. They have been cryo-sectioned from frozen, OCT-embedded tissue blocks to sections with 7 μm thickness and mounted in precise areas of a poly-l-lysine coated glass slide. The sections are then stored at −80 C. When ready to use, they are thawed, dried, fixed in 4% PFA, and permeabilized in a manner typical of standard tissue-staining methods. They are then imaged in a brightfield imaging system (e.g., Evos Fl Auto, Life Technologies) to enable alignment between detector elements and tissue morphology during downstream analysis.

Recovery of analytes with faithful spatial location reconstruction from each of the 5 ROIs of device 1400 is achieved in the following manner. First, first chip 1402 with 100 inlets, outlets, and channels with width ranging from 100 um near the ports to 20 um in the region of interest is clamped on top of the tissue section array, such that the horizontal portions of the serpentine channel manifold are located on their respective tissue sections. Then a first set of spatial barcodes is flowed through the channel manifold and binds to target analytes, such that analytes in tissue under the first row are given a barcode with one index, those in the second row are given a barcode with a second index, and so forth. Next, the sample is incubated to permit the probes to fully bind or interact with the analytes, and the first chip is removed and the sample array is washed to clear away unbound probes. Next, second chip 1404 with channels similar in dimensions to first chip 1402 is affixed to the sample array. Care is taken such that the vertical channel manifold of second chip 1404 interfaces with the tissue in the center of the areas of the tissue which had previously interfaced with the horizontal areas of first chip 1402. Again the regions of the tissue underneath each channel receive a barcode reflective of their position within the vertical channel manifold. Then the sample is incubated to permit ligation or other bridging between the first and second set of barcodes. At this stage there are 5×50×50=12,500 spatially-barcoded regions of tissue, with 2,500 unique barcodes, thus there are 5 repeat tissue elements for each spatial barcode. To remediate this ambiguity, the final chip, third chip 1408, with 5 windows corresponding to each of the 5 ROIs is firmly clamped to the tissue sample array using claim 1410. This permits physically-separated tissue dissociation and cellular/nuclear lysis of the 5 ROIs. The 5 separate lysates resulting from this collection step are prepared for NGS sequencing via a standard NGS library preparation protocol. During this protocol, 5 uniquely-indexed NGS primers are used. This unique primer acts as a third barcode, removing the 5-fold ambiguity introduced by the serpentine chip pair, and enabling subsequent pooling of the libraries from the 5 ROIs, with downstream analysis able to uniquely reconstruct the spatial location of each target analyte via interpretation of the three spatial barcodes attached to each analyte.

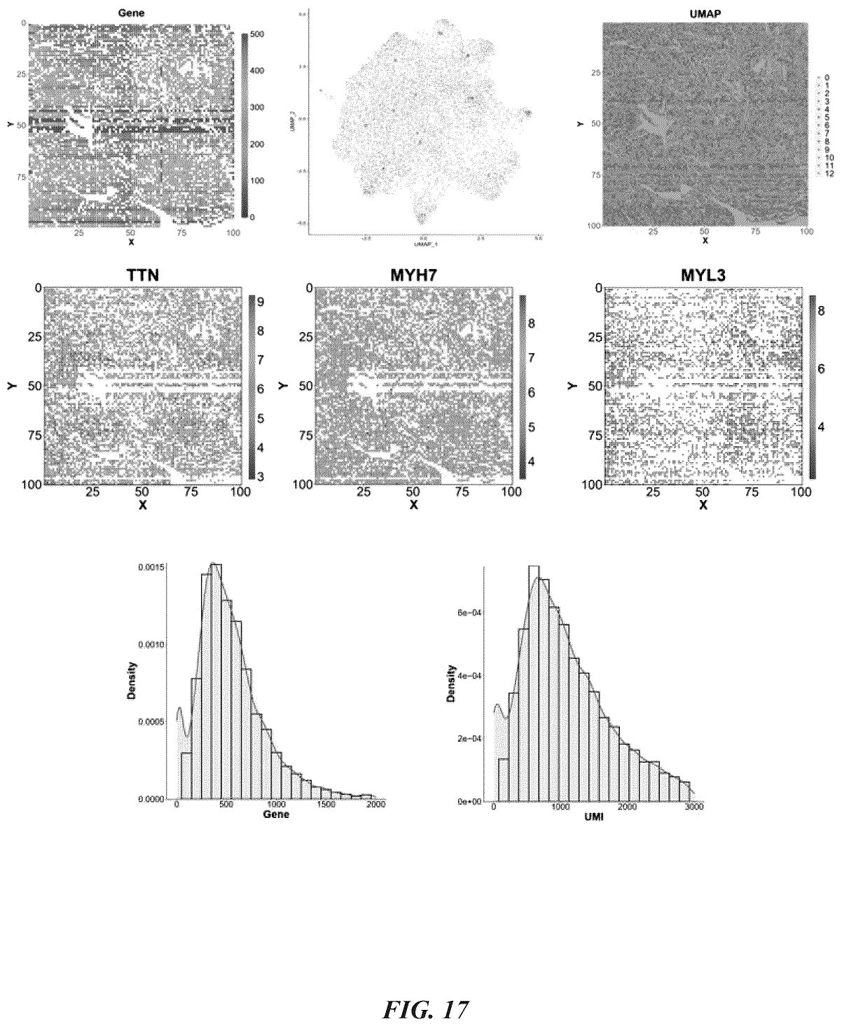

FIG. 16 depicts a series of data readouts describing the results of applying the above workflow to human heart section 1502, as shown in FIG. 15. From top-to-bottom, and left-to-right, the figures show: the pan-mRNA heatmap, where color intensity reflects the number of analytes recovered in each detector element as a function of geometrical location within the assayed tissue section; a plot in UMAP (Uniform Manifold Approximation and Projection) space showing cluster membership of each detector element as determined by the gene expression vectors measured in those detector elements; a plot by geometrical location in the tissue sample of the UMAP cluster membership; a heatmap showing the expression of mRNA corresponding to the genes “TTN”, “MYH7”, and “MYH11”; histograms showing the relative frequency of the number of unique genes and analytes recovered in each detector element.

FIG. 17 depicts the same plots as in FIG. 16, except for the bottom-most ROI (section 1504) in FIG. 15, instead of the top-most ROI.

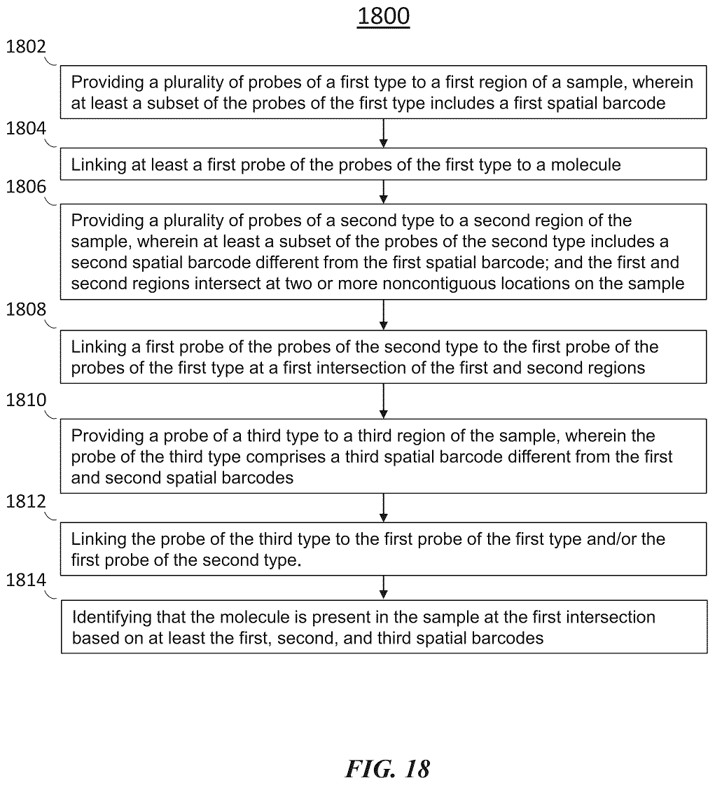

FIG. 18 is a flow diagram illustrating a method for a spatially encoded biological assay in accordance with some embodiments. Method 1800 can be performed using one or more of the devices and/or techniques illustrated in FIGS. 1, 2, 3, 5A, 5B, 8, 9A, 9B, 10A, 10B, 11A-11D, 12A, 12B, and/or 13A-13D.

As described below, method 1800 provides a flexible and scalable method for performing a spatially encoded biological assay.

At 1802, the method includes providing (e.g., by deposition, by reagent deposition, by microfluidic flow, by a microfluidic device, a microfluidic channel, microfluidic chip, polydimethylsiloxane microfluidic chip, microfluidic chip comprising at least 5, at least 10, at least 20, at least 30, at least 40, or at least 50 microchannels, 5 to 100 microchannels, 10 to 90 microchannels, 30 to 70 microchannels, 40 to 60 microchannels, or 50 microchannels, photoactivation (e.g., using a photomask)) a plurality of probes of a first type to a first region (e.g., a contiguous region, or a noncontiguous region) of a sample (e.g., a biological sample (for example, a murine (e.g., mouse or rat), feline (e.g., cat), canine (e.g., dog), equine (e.g., horse), bovine (e.g., cow), leporine (e.g., rabbit), porcine (e.g., pig), hircine (e.g., goat), ursine (e.g., bear), or piscine (e.g., fish) biological sample, a fixed biological sample (e.g., a sample fixed with a fixation agent selected from among formalin, formaldehyde, paraformaldehyde, and glutaraldehyde), a tissue (e.g., adult tissue, embryonic tissue, or fetal tissue), a tissue section, a tissue or tissue section that includes tumor cells, a cell, a bodily fluid (e.g., blood, urine, saliva, cerebrospinal fluid, or semen); in some embodiments the sample is mounted on a surface (e.g., a slide); a sample fixed before or after it is sectioned; in some embodiments, the fixation process involves perfusion of the animal from which the sample is collected; a formalin fixed paraffin-embedded (FFPE) tissue section). At least a subset of the probes of the first type includes (e.g., each includes) a first spatial barcode (e.g., a nucleic acid molecule or nucleic acid analog (e.g., 5 to 50 nucleotides (for example, 5 to 40, 5 to 30, 5 to 20, 5 to 10, 10 to 50, 10 to 40, 10 to 30, or 10 to 20 nucleotides, in some embodiments, 5, 10, 15, 20, 25, 30, 35, 40, 45, or 50 nucleotides)), a polynucleotide, an RNA molecule, a DNA molecule, a single-stranded nucleic acid molecule, a single-stranded RNA molecule, a single-stranded DNA molecule, a double-stranded nucleic acid molecule, a double-stranded RNA molecule, a chromogenic molecule, a fluorescent molecule, a polypeptide, a protein, a peptide, an antibody or fragment thereof, a whole antibody, an IgG antibody, an Fab antibody fragment thereof, an Fab′ antibody fragment, an F(ab′)2 antibody fragment, a monospecific Fab2 fragment, a bispecific Fab2 fragment, a trispecific Fab3 fragment, a single chain variable fragment (scFv), a bispecific diabody, a trispecific diabody, an Fc antibody fragment, an scFv-Fc molecule, a minibody, an enzyme, a ligand, a mass tag).

At 1804, the method includes linking (e.g., directly linking, indirectly linking, covalently linking, chemically linking, linking by click chemistry (e.g., cycloaddition, azide-alkyne cycloaddition, azide-cyclooctyne cycloaddition, azide-dibenzocyclooctyne cycloaddition, copper(I)-catalyzed azide-alkyne cycloaddition, Diels-Alder Reaction, inverse electron demand Diels-Alder Reaction, inverse electron demand Diels-Alder Reaction of a tetrazine and an olefin, inverse electron demand Diels-Alder Reaction of a tetrazine and an triazine, inverse electron demand Diels-Alder Reaction of a 1,2,4,5-tetrazine and an olefin, inverse electron demand Diels-Alder Reaction of a 1,2,4,5-tetrazine and a 1,2,3-Triazine, inverse electron demand Diels-Alder Reaction of a trans-cyclooctene and a tetrazine, inverse electron demand Diels-Alder Reaction of a trans-cyclooctene and a 1,2,4,5-tetrazine), linking by photoclick chemistry (e.g., 1,3-dipolar cycloaddition, tetrazole-alkene cycloaddition), noncovalently linking, linking by hydrogen bond, linking by base pair recognition of a complementary nucleic acid sequence, ligation, ligation with T4 ligase, ligation assisted with a ligation linker, ligation assisted with a universal ligation linker) at least a first probe of the probes of the first type to a molecule (e.g., a binder-tag conjugate (e.g., a protein-nucleic acid tag, an antibody-DNA tag (ADT)), a nucleic acid molecule, a nucleic acid analog, a polynucleotide, an RNA molecule, a DNA molecule, a single-stranded nucleic acid molecule, a single-stranded RNA molecule, an mRNA molecule, a single-stranded DNA molecule, a double-stranded nucleic acid molecule, a double-stranded RNA molecule, a chromogenic molecule, a fluorescent molecule, a polypeptide, a protein, a peptide, an antibody or fragment thereof, a whole antibody, an IgG antibody, an Fab antibody fragment thereof, an Fab′ antibody fragment, an F(ab′)2 antibody fragment, a monospecific Fab2 fragment, a bispecific Fab2 fragment, a trispecific Fab3 fragment, a single chain variable fragment (scFv), a bispecific diabody, a trispecific diabody, an Fc antibody fragment, an scFv-Fc molecule, a minibody, an enzyme, a ligand, a receptor, a lipid, a phospholipid, a triglyceride, a steroid, a carbohydrate, (e.g., a monosaccharaide, a disaccharide, a polysaccharide, a cytokine, a growth hormone) in the sample.

At 1806, the method includes providing a plurality of probes of a second type to a second region (e.g., a contiguous region, or a noncontiguous region) of the sample. At least a subset of the probes of the second type includes a second spatial barcode different from the first spatial barcode. Further, the first and second regions intersect at two or more (e.g., 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 25 or more, 30 or more, 35 or more, 40 or more, 45 or more, 50 or more, 60 or more, 70 or more, 80 or more, 90 or more, 100 or more, 150 or more, 200 or more, 250 or more, or 300 or more) noncontiguous locations on the sample.

At 1808, the method includes linking (e.g., in situ linking, directly linking, indirectly linking, covalently linking, chemically linking, linking by click chemistry (e.g., cycloaddition, azide-alkyne cycloaddition, azide-cyclooctyne cycloaddition, azide-dibenzocyclooctyne cycloaddition, copper(I)-catalyzed azide-alkyne cycloaddition, Diels-Alder Reaction, inverse electron demand Diels-Alder Reaction, inverse electron demand Diels-Alder Reaction of a tetrazine and an olefin, inverse electron demand Diels-Alder Reaction of a tetrazine and an triazine, inverse electron demand Diels-Alder Reaction of a 1,2,4,5-tetrazine and an olefin, inverse electron demand Diels-Alder Reaction of a 1,2,4,5-tetrazine and a 1,2,3-Triazine, inverse electron demand Diels-Alder Reaction of a trans-cyclooctene and a tetrazine, inverse electron demand Diels-Alder Reaction of a trans-cyclooctene and a 1,2,4,5-tetrazine), linking by photoclick chemistry (e.g., 1,3-dipolar cycloaddition, tetrazole-alkene cycloaddition), noncovalently linking, linking by hydrogen bond, linking by base pair recognition of a complementary nucleic acid sequence, ligation, ligation with T4 ligase, ligation assisted with a ligation linker, ligation assisted with a universal ligation linker) a first probe of the probes of the second type to the first probe of the probes of the first type at a first intersection (e.g., 10 micron by 10 micron, 25 micron by 25 micron) of the first and second regions.

At 1810, the method includes providing a probe of a third type (e.g., a plurality of probes of the third type) to a third region (e.g., third region comprising the first intersection of the first and second regions) of the sample, wherein the probe of the third type comprises a third spatial barcode different from the first and second spatial barcodes.

At 1812, the method includes linking the probe of the third type to the first probe of the first type and/or the first probe of the second type.

At 1814, the method includes identifying that the molecule is present in the sample at the first intersection based on (e.g., sequencing, next generation sequencing, mass spectrometry, light detection, fluorescence, mass spectrometry) at least the first, second, and third spatial barcodes.

In some embodiments, the method further includes linking a second probe of the probes of the first type and a second probe of the probes of the second type at a second intersection (e.g., 10 micron by 10 micron, 25 micron by 25 micron, first and second intersections are noncontiguous) of the first and second regions.

In some embodiments, the first and second regions intersect at three or more (e.g., 4 or more, 5 or more, 6 or more, 7 or more, 8 or more, 9 or more, 10 or more, 11 or more, 12 or more, 13 or more, 14 or more, 15 or more, 20 or more, 25 or more, 30 or more, 35 or more) noncontiguous locations on the sample.

In some embodiments, the third region includes the first intersection of the first and second regions.

In some embodiments, the area of the third region is at least about 10-fold (e.g., at least about 50-fold, 100-fold, 150-fold, 200-fold, 300-fold, 400-fold, 500-fold, 600-fold, 700-fold, 800-fold, 900-fold, 1000-fold, 1500-fold, 2000-fold, 2500-fold, 3000-fold, 3500-fold, 4000-fold, 4500-fold, 5000-fold, 6000-fold, 7000-fold, 8000-fold, 9000-fold, or 10000-fold) larger than the area of the first intersection of the first and second regions.

In some embodiments, the probes of the first type and the probes of the second type are provided by microfluidic flow, and the probe of the third type is provided by reagent deposition.

In some embodiments, the probes of the first type, the probes of the second type, and the probe of the third type are provided by microfluidic flow.

In some embodiments, the microfluidic flow of the probe of the third type is at least about 2-fold (e.g., at least about 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 60-fold, 70-fold, 80-fold, 90-fold, 100-fold, 150-fold, 200-fold, 300-fold) wider than the width of the microfluidic flow of the probes of the first type and the probes of the second type.

In some embodiments, the method further includes identifying that the molecule is present at an intersection of the first, second, and third regions.

In some embodiments, a plurality of probes of the probes of the third type are provided, wherein at least a subset of the probes of the third type includes a third spatial barcode different from the first and second spatial barcodes.

In some embodiments, a probe of the third type is provided to two or more (e.g., 3 or more, 4 or more, 5 or more, 6 or more, 7 or more, 8 or more, 9 or more, 10 or more, 11 or more, 12 or more, 13 or more, 14 or more, 15 or more, 20 or more, 25 or more, 30 or more, 35 or more) intersections of the first and second regions.

In some embodiments, the method further includes providing a probe of a fourth type (e.g., a plurality of probes of the fourth type) to a fourth region (e.g., fourth region comprising the first intersection of the first and second regions) of the sample, wherein the probe of the fourth type comprises a fourth spatial barcode different from the first, second and third spatial barcodes. In such embodiments, the method further includes linking the probe of the fourth type to one or more of the first probe of the first type, first probe of the second type, and probe of the third type. In such embodiments, the method further includes identifying that the molecule is present in the sample at the first intersection of the first and second regions based on at least the first, second, third and fourth spatial barcodes.

In some embodiments, the method further includes identifying that the molecule is present at an intersection of the first, second, third, and fourth regions.

In some embodiments, the first, second, third, and fourth probes are provided by microfluidic flow.

In some embodiments, the microfluidic flow of the probe of the third type is at least about 2-fold (e.g., at least about 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 60-fold, 70-fold, 80-fold, 90-fold, 100-fold, 150-fold, 200-fold, 300-fold) wider than the width of the microfluidic flow of the probes of the first type and the probes of the second type. In such embodiments, the microfluidic flow of the probe of the fourth type is at least about 2-fold (e.g., at least about 3-fold, 4-fold, 5-fold, 6-fold, 7-fold, 8-fold, 9-fold, 10-fold, 15-fold, 20-fold, 25-fold, 30-fold, 35-fold, 40-fold, 45-fold, 50-fold, 60-fold, 70-fold, 80-fold, 90-fold, 100-fold, 150-fold, 200-fold, 300-fold) wider than the width of the microfluidic flow of the probes of the first type and the probes of the second type.

In some embodiments, the first probe of the first type comprises a first molecular probe region (e.g., a nucleic acid molecule, a nucleic acid analog, a polynucleotide, an RNA molecule, a DNA molecule, a single-stranded nucleic acid molecule, a single-stranded RNA molecule, a single-stranded DNA molecule (e.g., comprising a polyT sequence (for example, having a length of 5 to 50 nucleotides (e.g., 5 to 40, 5 to 30, 5 to 20, 5 to 10, 10 to 50, 10 to 40, 10 to 30, or 10 to 20 nucleotides), in some embodiments, a polyT sequence may have a length of 5, 10, 15, 20, 25, 30, 35, 40, 45, or 50 nucleotides)), a double-stranded nucleic acid molecule, a double-stranded RNA molecule, a polypeptide, a protein, a peptide, an antibody or fragment thereof, a whole antibody, an IgG antibody, an Fab antibody fragment thereof, an Fab′ antibody fragment, an F(ab′)₂ antibody fragment, a monospecific Fab₂ fragment, a bispecific Fab₂ fragment, a trispecific Fab₃ fragment, a single chain variable fragment (scFv), a bispecific diabody, a trispecific diabody, an Fc antibody fragment, an scFv-Fc molecule, a minibody, an enzyme, a ligand, a receptor, a lipid, a phospholipid, a triglyceride, a steroid, a carbohydrate (e.g., a monosaccharaide, a disaccharide, a polysaccharide, a cytokine, a growth hormone, a photocleavable molecule)), and linking the first probe of the first type to the molecule comprises linking the first molecular probe region to the molecule.

In some embodiments, the molecule is mRNA; the first molecular probe region includes a polyT sequence. In such embodiments, the method further includes producing cDNA linked to the first probe by a reverse transcription reaction.

In some embodiments, the method further includes identifying the molecule.

In some embodiments, the molecule is an mRNA molecule or a binder-tag conjugate (e.g., a protein-nucleic acid tag, an antibody-DNA tag (ADT)).

In some embodiments, the molecule is a binder-tag conjugate (e.g., a protein-nucleic acid tag, an antibody-DNA tag (ADT)), and the method further comprises providing the binder-tag conjugate to the sample.

In some embodiments, at least one probe of the probes of the first type, probes of the second type, or probe of the third type comprises a universal molecular identifier (UMI) (e.g., a nucleic acid or nucleic acid analog, (e.g., 5 to 50 nucleotides, 5 to 40 nucleotides, 5 to 30 nucleotides, 5 to 20 nucleotides, 5 to 10 nucleotides, 10 to 50 nucleotides, 10 to 40 nucleotides, 10 to 30 nucleotides, 10 to 20 nucleotides, 5 or more nucleotides, 10 or more nucleotides, 20 or more nucleotides, 30 or more nucleotides, 40 or more nucleotides, 50 or more nucleotides), DNA, RNA).

In some embodiments, at least one probe of the probes of the first type, probes of the second type, or probe of the third type comprises a ligation linker (e.g., a nucleic acid or nucleic acid analog, (e.g., 5 to 50 nucleotides, 5 to 40 nucleotides, 5 to 30 nucleotides, 5 to 20 nucleotides, 5 to 10 nucleotides, 10 to 50 nucleotides, 10 to 40 nucleotides, 10 to 30 nucleotides, 10 to 20 nucleotides, 5 or more nucleotides, 10 or more nucleotides, 20 or more nucleotides, 30 or more nucleotides, 40 or more nucleotides, 50 or more nucleotides), DNA, RNA).

In some embodiments, the first, second, and third spatial barcodes include nucleic acid molecules or nucleic acid analog molecules. In such embodiments, identifying that the molecule is present in the sample at the first intersection comprises sequencing (e.g., next-generation sequencing, chain termination sequencing, pyrosequencing) the nucleic acid molecules of the first, second, and third spatial barcodes.

In some embodiments, the first probe of the first type is directly or indirectly linked to the molecule in the sample.

In some embodiments, linking the at least one of the first probes to the molecule includes photoactivation (e.g., photoactivation of a photo-activateable oligonucleotide (e.g., wherein photoactivation results in release of a blocking agent that prevents the first probe from linking to the molecule), photoactivation of a transcriptome in vivo analysis tag (TIVA tag), photoactivation of a 1,3-dipolar cycloaddition, photoactivation of a tetrazole-alkene cycloaddition; photoactivation at the first region; using a photomask).

In some embodiments, linking the at least one of the first probes to the molecule comprises photoactivation of the first probe.

In some embodiments, linking the first and second probes includes photoactivation (e.g., . . . photoactivation at the second region), and linking the third probe to the first and/or second probe comprises photoactivation (e.g., . . . photoactivation at the third region).

In some embodiments, linking the first and second probes includes photoactivation of the second probe, and linking the third probe to the first and/or second probe comprises photoactivation of the third probe.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the claims.

What is claimed is:

1. A microfluidic-based method for mapping spatial distribution of molecules in a sample, the method comprising:
providing a plurality of probes having a first spatial barcode to a first region of the sample, via a microfluidic channel of a first device;
linking at least a first probe having the first spatial barcode of the plurality of probes having the first spatial barcode to a molecule in the sample;
providing a plurality of probes having a second spatial barcode, different from the first spatial barcode, to a second region of the sample, via a microfluidic channel of a second device, wherein the first and second regions intersect at two or more noncontiguous locations on the sample;
linking a first probe having the second spatial barcode of the plurality of probes having the second spatial barcode to the first probe having the first spatial barcode at a first intersection of the first and second regions;
providing a plurality of probes having a third spatial barcode, different from the first and second spatial barcodes, to a third region of the sample, via a third device;
linking a first probe having the third spatial barcode of the plurality of probes having the third spatial barcode to the first probe having the first spatial barcode and/or the first probe having the second spatial barcode; and
identifying that the molecule is present in the sample at the first intersection based on at least the first, second, and third spatial barcodes.

2. The microfluidic-based method of claim 1, wherein the method further comprises linking a second probe of the plurality of probes having the first spatial barcode and a second probe of the plurality of probes of the plurality of probes having the second spatial barcode at a second intersection of the first and second regions.

3. The microfluidic-based method of claim 1, wherein the first and second regions intersect at four noncontiguous locations on the sample.

4. The microfluidic-based method of claim 1, wherein the third region comprises the first intersection of the first and second regions.

5. The microfluidic-based method of claim 1, wherein the area of the third region is at least 10-fold larger than the area of the first intersection of the first and second regions.

6. The microfluidic-based method of claim 1, wherein the plurality of probes having the third spatial barcode are provided by reagent deposition.

7. The microfluidic-based method of claim 1, the plurality of probes having the third spatial barcode are provided by microfluidic flow.

8. The microfluidic-based method of claim 7, wherein the microfluidic flow of the plurality of probes having the third spatial barcode is at least 2-fold wider than a width of the microfluid channel of the first device.

9. The microfluidic-based method of claim 1, further comprising identifying that the molecule is present at an intersection of the first, second, and third regions.

10. The microfluidic-based method of claim 1, wherein a second plurality of probes having the third spatial barcode are provided to two or more intersections of the first and second regions.

11. The microfluidic-based method of claim 1, wherein the method further comprises:
providing a probe, to a fourth region of the sample having a fourth spatial barcode different from the first, second and third spatial barcodes;
linking the probe having the fourth spatial barcode to one or more of the first probe having the first spatial barcode, the first probe having the second spatial barcode, and the third probe having the third spatial barcode; and
identifying that the molecule is present in the sample at the first intersection of the first and second regions based on at least the first, second, third, and fourth spatial barcodes.

12. The microfluidic-based method of claim 11, further comprising identifying that the molecule is present at an intersection of the first, second, third, and fourth regions.

13. The microfluidic-based method of claim 11, the probe having the fourth spatial barcode is provided by microfluidic flow.

14. The microfluidic-based method of claim 13, wherein the microfluidic flow of the probe having the fourth spatial barcode is at least 2-fold wider than a width of the microfluid channel of the first device.

15. The microfluidic-based method of claim 1, wherein the first probe having the first spatial barcode comprises a first molecular probe region, and linking the first probe having the first spatial barcode to the molecule comprises linking the first molecular probe region to the molecule.

16. The microfluidic-based method of claim 15, wherein:
the molecule is mRNA;
the first molecular probe region comprises a polyT sequence; and
the method further comprises producing cDNA linked to the first probe by a reverse transcription reaction.

17. The microfluidic-based method of claim 1, wherein the method further comprises identifying the molecule.

18. The microfluidic-based method of claim 1, wherein the molecule is nucleic acid molecule.

19. The microfluidic-based method of claim 1, wherein the molecule is a binder-tag conjugate, and the method further comprises providing the binder-tag conjugate to the sample.

20. The microfluidic-based method of claim 1, wherein at least one probe of the of the plurality of probes having the first spatial barcode comprises a universal molecular identifier (UMI).

21. The microfluidic-based method of claim 1, wherein at least one probe of the plurality of probes having the second barcode comprises a ligation linker.

22. The microfluidic-based method of claim 1, wherein:
the first, second, and third spatial barcodes comprise nucleic acid molecules; and
identifying that the molecule is present in the sample at the first intersection comprises sequencing the nucleic acid molecules of the first, second, and third spatial barcodes.

23. The microfluidic-based method of claim 1, wherein the first probe having the first spatial barcode is directly linked to the molecule in the sample.

24. The microfluidic-based method of claim 1, wherein linking the first probe having the first spatial barcode to the molecule comprises photoactivation.

25. The microfluidic-based method of claim 1, wherein:
linking the first probe having the second spatial barcode to the first probe having the first spatial barcode comprises photoactivation; and
linking the first probe having the third spatial barcode to the first probe having the first spatial barcode and/or to the first probe having the second spatial barcode comprises photoactivation.
